SEQUENCE LISTING (276 sequences)

SEQ ID NO: 1 - DNA, 18 bp, Anas platyrhynchos
ctcctactggctatgcac

SEQ ID NO: 2 - DNA, 19 bp, Anas platyrhynchos
gtaatcctactgctcactc

SEQ ID NO: 3 - DNA, 38 bp, Anas platyrhynchos
ttcggatctctgctcgccatctgcctggccacacaaat

SEQ ID NO: 4 - DNA, 24 bp, Anas platyrhynchos
gacacatcccttgctttctcctca

SEQ ID NO: 5 - DNA, 33 bp, Anser anser
ctcccttctagccatctgcttagccacacaaat

SEQ ID NO: 6 - DNA, 21 bp, Anser anser
ccgcagacacttcactcgcct

SEQ ID NO: 7 - DNA, 25 bp, Anser anser
caacggtgcttcgctcttctttatc

SEQ ID NO: 8 - DNA, 18 bp, Anser anser
cacttcactcgccttctc

SEQ ID NO: 9 - DNA, 16 bp, Cairina moschata
aacctgcacgccaatg

SEQ ID NO: 10 - DNA, 35 bp, Cairina moschata
gggtccctcctcgccatttgcctggtcacccaaat

SEQ ID NO: 11 - DNA, 17 bp, Cairina moschata
gtcctgccatggggaca

SEQ ID NO: 12 - DNA, 22 bp, Cairina moschata
ctcctactcgccctcatggcaa

SEQ ID NO: 13 - DNA, 20 bp, Cairina moschata
atccgcaacctgcacgccaa

SEQ ID NO: 14 - DNA, 24 bp, Cairina moschata
tcctcagtggctaacacatgtcga

SEQ ID NO: 15 - DNA, 17 bp, Rangifer tarandus
cgagacgtcaattatgg

SEQ ID NO: 16 - DNA, 17 bp, Rangifer tarandus
atctgcttatttataca

SEQ ID NO: 17 - DNA, 17 bp, Rangifer tarandus
tcctctgttactcacat

SEQ ID NO: 18 - DNA, 17 bp, Rangifer tarandus
tcctcttatttacagta

SEQ ID NO: 19 - DNA, 27 bp, Rangifer tarandus
aatattggagtgatcctcttatttaca

SEQ ID NO: 20 - DNA, 17 bp, Columba palumbus
acacaggagtcgtcctc

SEQ ID NO: 21 - DNA, 16 bp, Columba palumbus
ttgctaactcaaatcc

SEQ ID NO: 22 - DNA, 17 bp, Columba palumbus
acccttatagccactgc

SEQ ID NO: 23 - DNA, 23 bp, Columba palumbus
ggcttactactcgccgcacatta

SEQ ID NO: 24 - DNA, 17 bp, Columba palumbus
ctaaccggcttactact

SEQ ID NO: 25 - DNA, 23 bp, Columba palumbus
ggcatttgcttgctaactcaaat

SEQ ID NO: 26 - DNA, 19 bp, Acipenser baerii
ctcactcataggcctctgc

SEQ ID NO: 27 - DNA, 17 bp, Acipenser baerii
tggctcactcataggcc

SEQ ID NO: 28 - DNA, 17 bp, Coturnix coturnix
ctgcttctcacactaat

SEQ ID NO: 29 - DNA, 16 bp, Coturnix coturnix
tcaccggccttctact

SEQ ID NO: 30 - DNA, 16 bp, Coturnix coturnix
tagcaatatgcctcat

SEQ ID NO: 31 - DNA, 21 bp, Sardina pilchardus
cttcggatcgcttcttggcct

SEQ ID NO: 32 - DNA, 24 bp, Sardina pilchardus
ctccttcttttggtcatgataact

SEQ ID NO: 33 - DNA, 20 bp, Sardina pilchardus
gggcgagggctctattatgg

SEQ ID NO: 34 - DNA, 17 bp, Sardina pilchardus
attgggcgagggctcta

SEQ ID NO: 35 - DNA, 20 bp, Sardina pilchardus
gttgtcctccttcttttggt

SEQ ID NO: 36 - DNA, 16 bp, Sardina pilchardus
atggagcatctttttt

SEQ ID NO: 37 - DNA, 17 bp, Sardina pilchardus
ttggttatgtcttaccg

SEQ ID NO: 38 - DNA, 48 bp, Sardina pilchardus
tggcctctgtctagcggcccagattctgacagggttgttcttagccat

SEQ ID NO: 39 - DNA, 21 bp, Sardina pilchardus
tgattcgaagtatgcacgcaa

SEQ ID NO: 40 - DNA, 17 bp, Sardina pilchardus
tttgtatttacgcccac

SEQ ID NO: 41 - DNA, 19 bp, Sardina pilchardus
cctctgacatcgcaaccgc

SEQ ID NO: 42 - DNA, 19 bp, Anguilla anguilla
atacctttacatagaaaca

SEQ ID NO: 43 - DNA, 16 bp, Gallus gallus
gtgggctatgttctcc

SEQ ID NO: 44 - DNA, 18 bp, Gallus gallus
tccctattagcagtctgc

SEQ ID NO: 45 - DNA, 19 bp, Gallus gallus
tcatccggaatctccacgc

SEQ ID NO: 46 - DNA, 21 bp, Gallus gallus
catctgtatcttccttcacat

SEQ ID NO: 47 - DNA, 23 bp, Gallus gallus
gtagcccacacttgccggaacgt

SEQ ID NO: 48 - DNA, 17 bp, Scomber japonicus
ggacttttcctcgcaat

SEQ ID NO: 49 - DNA, 23 bp, Scomber japonicus
tgcctaatttctcaaattctcac

SEQ ID NO: 50 - DNA, 20 bp, Scomber japonicus
ttcggctcactgcttggtct

SEQ ID NO: 51 - DNA, 20 bp, Scomber japonicus
cactacacccccgatgttga

SEQ ID NO: 52 - DNA, 25 bp, Scomber japonicus
tcctaccttttcatggaaacatgaa

SEQ ID NO: 53 - DNA, 36 bp, Scomber japonicus
acccccgatgttgagtcagcattcgactcagtcgcc

SEQ ID NO: 54 - DNA, 18 bp, Anguilla japonica
tatggatgattcatccga

SEQ ID NO: 55 - DNA, 21 bp, Anguilla japonica
gatgattcatccgaaatttac

SEQ ID NO: 56 - DNA, 17 bp, Anguilla japonica
ataataactgcattcgt

SEQ ID NO: 57 - DNA, 19 bp, Meleagris gallopavo
tattatggttcgtacctat

SEQ ID NO: 58 - DNA, 17 bp, Meleagris gallopavo
aacctccatgcgaatgg

SEQ ID NO: 59 - DNA, 26 bp, Meleagris gallopavo
gcagacaccactcttgcattctcttc

SEQ ID NO: 60 - DNA, 27 bp, Meleagris gallopavo
ttctcttctgtggcctacacatgccga

SEQ ID NO: 61 - DNA, 17 bp, Meleagris gallopavo
tgcctcatcactcaaat

SEQ ID NO: 62 - DNA, 18 bp, Meleagris gallopavo
cttaaccggcctcctact

SEQ ID NO: 63 - DNA, 28 bp, Meleagris gallopavo
caggagtagtcttacttctcaccctcat

SEQ ID NO: 64 - DNA, 18 bp, Meleagris gallopavo
ctcatcactcaaatctta

SEQ ID NO: 65 - DNA, 16 bp, Scomber scombrus
ctcctcgtaatgatga

SEQ ID NO: 66 - DNA, 17 bp, Scomber scombrus
ttccttgcaatgcacta

SEQ ID NO: 67 - DNA, 19 bp, Scomber scombrus
atgaaacgtcggtgtagtc

SEQ ID NO: 68 - DNA, 17 bp, Scomber scombrus
ggtgtagtcctcctcct

SEQ ID NO: 69 - DNA, 19 bp, Scomber scombrus
tcatccgcaacatgcacgc

SEQ ID NO: 70 - DNA, 33 bp, Scomber scombrus
tacacgcccgacgtcgaatcagcattcaactca

SEQ ID NO: 71 - DNA, 17 bp, Scomber scombrus
ggttccctgcttggtct

SEQ ID NO: 72 - DNA, 17 bp, Anguilla mossambica
aatggagcttctttctt

SEQ ID NO: 73 - DNA, 26 bp, Anguilla mossambica
ggactatgtcttatctctcaaatcct

SEQ ID NO: 74 - DNA, 20 bp, Canis familiaris
tatccgctatatgcacgcaa

SEQ ID NO: 75 - DNA, 21 bp, Canis familiaris
ggagtatgcttgattctacag

SEQ ID NO: 76 - DNA, 18 bp, Canis familiaris
cggatcctatgtattcat

SEQ ID NO: 77 - DNA, 24 bp, Canis familiaris
acattggaattgtactattattcg

SEQ ID NO: 78 - DNA, 16 bp, Canis familiaris
actattattcgcaacc

SEQ ID NO: 79 - DNA, 16 bp, Canis familiaris
attatccgctatatgc

SEQ ID NO: 80 - DNA, 16 bp, Canis familiaris
caggtttattcttagc

SEQ ID NO: 81 - DNA, 16 bp, Canis familiaris
gcaaccatagccacag

SEQ ID NO: 82 - DNA, 18 bp, Canis familiaris
aaatggcgcttccatatt

SEQ ID NO: 83 - DNA, 16 bp, Canis familiaris
taggagtatgcttgat

SEQ ID NO: 84 - DNA, 16 bp, Numida meleagris
gacccaaattatcacc

SEQ ID NO: 85 - DNA, 19 bp, Numida meleagris
atccctcctagcagtctgc

SEQ ID NO: 86 - DNA, 16 bp, Numida meleagris
atgacccaaattatca

SEQ ID NO: 87 - DNA, 18 bp, Numida meleagris
tgtcgaaatgtccaatac

SEQ ID NO: 88 - DNA, 18 bp, Equus asinus
agacactacaactgcctt

SEQ ID NO: 89 - DNA, 16 bp, Equus asinus
gctcctacacattcct

SEQ ID NO: 90 - DNA, 17 bp, Equus asinus
atcagacactacaactg

SEQ ID NO: 91 - DNA, 18 bp, Equus asinus
tgcctctttatccacgta

SEQ ID NO: 92 - DNA, 16 bp, Auxis thazard
ttggcgtagttcttct

SEQ ID NO: 93 - DNA, 29 bp, Equus caballus
cagatgaattatccaccatctccatgcta

SEQ ID NO: 94 - DNA, 23 bp, Equus caballus
atgtgaactacagatgaattatc

SEQ ID NO: 95 - DNA, 25 bp, Equus caballus
ttctcctatttcttccagtaatagc

SEQ ID NO: 96 - DNA, 23 bp, Equus caballus
tcctagctatatactacacatca

SEQ ID NO: 97 - DNA, 25 bp, Equus caballus
gaaatattgggattctcctatttct

SEQ ID NO: 98 - DNA, 18 bp, Equus caballus
gccttctttggttccctc

SEQ ID NO: 99 - DNA, 22 bp, Equus caballus
tctcatctgttatacacatctg

SEQ ID NO: 100 - DNA, 23 bp, Equus caballus
tcacgtaggacaaggcctttact

SEQ ID NO: 101 - DNA, 23 bp, Equus caballus
gcctttactacagctcctacacc

SEQ ID NO: 102 - DNA, 21 bp, Equus caballus
ctttggttcccacctaggaat

SEQ ID NO: 103 - DNA, 16 bp, Equus caballus
tcccacctaggaatct

SEQ ID NO: 104 - DNA, 19 bp, Equus caballus
tgcctctttattcacgtag

SEQ ID NO: 105 - DNA, 17 bp, Euthynnus alletteratus
attggtgtagtacttct

SEQ ID NO: 106 - DNA, 17 bp, Euthynnus alletteratus
tttgcatttactcacac

SEQ ID NO: 107 - DNA, 17 bp, Euthynnus alletteratus
ggcctgttcctcgcaat

SEQ ID NO: 108 - DNA, 16 bp, Euthynnus alletteratus
gcatttactcacacat

SEQ ID NO: 109 - DNA, 17 bp, Xiphias gladius
tatgtattaccctgagg

SEQ ID NO: 110 - DNA, 30 bp, Xiphias gladius
gacatcgcgacggcctttacatccgtagca

SEQ ID NO: 111 - DNA, 16 bp, Xiphias gladius
ccctcctcggcctctg

SEQ ID NO: 112 - DNA, 21 bp, Xiphias gladius
ggcctgtttctcgctatacac

SEQ ID NO: 113 - DNA, 29 bp, Xiphias gladius
tctgtttagctgcccaagtcctcacaggc

SEQ ID NO: 114 - DNA, 17 bp, Xiphias gladius
ctcggcctctgtttagc

SEQ ID NO: 115 - DNA, 17 bp, Xiphias gladius
tcctatctatacaaaga

SEQ ID NO: 116 - DNA, 19 bp, Xiphias gladius
catcagacatcgcgacggc

SEQ ID NO: 117 - DNA, 16 bp, Gadus morhua
tgactaattcggaata

SEQ ID NO: 118 - DNA, 20 bp, Gadus morhua
catgctaatggtgcctcttt

SEQ ID NO: 119 - DNA, 17 bp, Gadus morhua
ggttcctatctttttgt

SEQ ID NO: 120 - DNA, 17 bp, Phasianus colchicus
aaacactggagtcgtcc

SEQ ID NO: 121 - DNA, 16 bp, Phasianus colchicus
gaaatgtgcagtacgg

SEQ ID NO: 122 - DNA, 20 bp, Phasianus colchicus
ggttccctgctagcagtatg

SEQ ID NO: 123 - DNA, 18 bp, Phasianus colchicus
actggcctcctattagcc

SEQ ID NO: 124 - DNA, 17 bp, Phasianus colchicus
tgccttattactcaaat

SEQ ID NO: 125 - DNA, 18 bp, Phasianus colchicus
tgtcgaaatgtgcagtac

SEQ ID NO: 126 - DNA, 17 bp, Struthio camelus
accggcgttatcctcct

SEQ ID NO: 127 - DNA, 20 bp, Struthio camelus
tgaaacaccggcgttatcct

SEQ ID NO: 128 - DNA, 18 bp, Struthio camelus
ttttggatcgctactagg

SEQ ID NO: 129 - DNA, 24 bp, Struthio camelus
cagtacggatgatttatccgcaat

SEQ ID NO: 130 - DNA, 17 bp, Struthio camelus
cacacatgccggaacgt

SEQ ID NO: 131 - DNA, 23 bp, Struthio camelus
tcctactaacattaatagcaact

SEQ ID NO: 132 - DNA, 16 bp, Struthio camelus
aattttggatcgctac

SEQ ID NO: 133 - DNA, 20 bp, Struthio camelus
ctaacagggctcctactagc

SEQ ID NO: 134 - DNA, 16 bp, Struthio camelus
cacagccgacactaca

SEQ ID NO: 135 - DNA, 18 bp, Felis catus
ctgtcgcgacgttaatta

SEQ ID NO: 136 - DNA, 23 bp, Felis catus
cctacaccttctcagagacatga

SEQ ID NO: 137 - DNA, 21 bp, Felis catus
tatctgcctgtacatacatgt

SEQ ID NO: 138 - DNA, 17 bp, Felis catus
attggaatcatactatt

SEQ ID NO: 139 - DNA, 23 bp, Felis catus
acagcttttatgggatacgtcct

SEQ ID NO: 140 - DNA, 25 bp, Felis catus
caccggcctctttttggccatacac

SEQ ID NO: 141 - DNA, 25 bp, Felis catus
ggaatcatactattatttacagtca

SEQ ID NO: 142 - DNA, 22 bp, Homo sapiens
accagacgcctcaaccgccttt

SEQ ID NO: 143 - DNA, 23 bp, Homo sapiens
tcctcctgcttgcaactatagca

SEQ ID NO: 144 - DNA, 33 bp, Homo sapiens
ctcactccttggcgcctgcctgatcctccaaat

SEQ ID NO: 145 - DNA, 20 bp, Homo sapiens
tccaaatcaccacaggacta

SEQ ID NO: 146 - DNA, 20 bp, Homo sapiens
atcgcccacatcactcgaga

SEQ ID NO: 147 - DNA, 17 bp, Homo sapiens
ctcaccagacgcctcaa

SEQ ID NO: 148 - DNA, 29 bp, Homo sapiens
ttacggatcatttctctactcagaaacct

SEQ ID NO: 149 - DNA, 18 bp, Homo sapiens
atctgcctcttcctacac

SEQ ID NO: 150 - DNA, 16 bp, Homo sapiens
ccatgcactactcacc

SEQ ID NO: 151 - DNA, 17 bp, Homo sapiens
tcctccaaatcaccaca

SEQ ID NO: 152 - DNA, 17 bp, Gadus ogac
catgctaacggtgcctc

SEQ ID NO: 153 - DNA, 20 bp, Gadus ogac
tttttatttgtctctatata

SEQ ID NO: 154 - DNA, 19 bp, Gadus ogac
tttgtctctatatacatat

SEQ ID NO: 155 - DNA, 18 bp, Bison bison
cttctacttacagtaata

SEQ ID NO: 156 - DNA, 18 bp, Bison bison
cgggtcttataccttcct

SEQ ID NO: 157 - DNA, 17 bp, Lepus europaeus
tcctaactggcttattt

SEQ ID NO: 158 - DNA, 23 bp, Lepus europaeus
ggctctctattgggattatgcct

SEQ ID NO: 159 - DNA, 18 bp, Lepus europaeus
aataatccagatcctaac

SEQ ID NO: 160 - DNA, 16 bp, Lepus europaeus
ctaataatccagatcc

SEQ ID NO: 161 - DNA, 22 bp, Lepus europaeus
gactcattcgttacttacacgc

SEQ ID NO: 162 - DNA, 26 bp, Euthynnus pelamis
tatacccctgacgtagaatcagcctt

SEQ ID NO: 163 - DNA, 19 bp, Euthynnus pelamis
atttactcccatattggcc

SEQ ID NO: 164 - DNA, 18 bp, Euthynnus pelamis
ctgcatttactcccatat

SEQ ID NO: 165 - DNA, 16 bp, Macropus giganteus
attctttatatgccta

SEQ ID NO: 166 - DNA, 16 bp, Macropus giganteus
tctttatatgcctatt

SEQ ID NO: 167 - DNA, 16 bp, Macropus giganteus
ctttggctcgctacta

SEQ ID NO: 168 - DNA, 16 bp, Macropus giganteus
ttggctcgctactagg

SEQ ID NO: 169 - DNA, 16 bp, Macropus giganteus
atattctttatatgcc

SEQ ID NO: 170 - DNA, 20 bp, Merluccius merluccius
ctatttctagcgatacatta

SEQ ID NO: 171 - DNA, 23 bp, Merluccius merluccius
tcctacttattcatagagacctg

SEQ ID NO: 172 - DNA, 17 bp, Merluccius merluccius
aacggcgcttctttctt

SEQ ID NO: 173 - DNA, 24 bp, Merluccius merluccius
aggcctctgcttagccgcccaaat

SEQ ID NO: 174 - DNA, 22 bp, Merluccius merluccius
ctcatccgtcgtacacatctgc

SEQ ID NO: 175 - DNA, 23 bp, Merluccius merluccius
ggagttgtactattccttttagt

SEQ ID NO: 176 - DNA, 19 bp, Merluccius merluccius
ttagccgcccaaatcttaa

SEQ ID NO: 177 - DNA, 34 bp, Merluccius merluccius
cattataccgcaaacgtcgagatagctttctcat

SEQ ID NO: 178 - DNA, 16 bp, Bos taurus
tcaatgttttttatct

SEQ ID NO: 179 - DNA, 17 bp, Bos taurus
tcctctgttacccatat

SEQ ID NO: 180 - DNA, 24 bp, Bos taurus
gtaatccttctgctcacagtaata

SEQ ID NO: 181 - DNA, 17 bp, Macropus rufus
ggctcatatctctacaa

SEQ ID NO: 182 - DNA, 17 bp, Macropus rufus
aggagcctgcttaatta

SEQ ID NO: 183 - DNA, 16 bp, Macropus rufus
gattgatccgcaatct

SEQ ID NO: 184 - DNA, 16 bp, Macropus rufus
tacggctgattgatcc

SEQ ID NO: 185 - DNA, 16 bp, Oncorhynchus mykiss
gtttgccacatctgcc

SEQ ID NO: 186 - DNA, 17 bp, Oncorhynchus mykiss
ctatgtttagctaccca

SEQ ID NO: 187 - DNA, 20 bp, Oncorhynchus mykiss
tatacctccgacatttcaac

SEQ ID NO: 188 - DNA, 16 bp, Oncorhynchus mykiss
cctggaatatcggagt

SEQ ID NO: 189 - DNA, 16 bp, Oncorhynchus mykiss
tcattcgaaacatcca

SEQ ID NO: 190 - DNA, 19 bp, Oncorhynchus mykiss
ttgtacttttacttctcac

SEQ ID NO: 191 - DNA, 16 bp, Oncorhynchus mykiss
gctcgtacctctacaa

SEQ ID NO: 192 - DNA, 17 bp, Oncorhynchus mykiss
gagttgtacttttactt

SEQ ID NO: 193 - DNA, 20 bp, Oncorhynchus mykiss
cgagatgttagttacggctg

SEQ ID NO: 194 - DNA, 18 bp, Mus musculus
gtacttctactgttcgca

SEQ ID NO: 195 - DNA, 16 bp, Mus musculus
caggtcttttcttagc

SEQ ID NO: 196 - DNA, 17 bp, Mus musculus
tttgggtcccttctagg

SEQ ID NO: 197 - DNA, 21 bp, Mus musculus
gtctgcctaatagtccaaatc

SEQ ID NO: 198 - DNA, 21 bp, Mus musculus
atcattacaggtcttttctta

SEQ ID NO: 199 - DNA, 17 bp, Mus musculus
ttccttcatgtcggacg

SEQ ID NO: 200 - DNA, 18 bp, Mus musculus
taatagtccaaatcatta

SEQ ID NO: 201 - DNA, 16 bp, Mus musculus
attggagtacttctac

SEQ ID NO: 202 - DNA, 16 bp, Salmo salar
gagttgtacttctact

SEQ ID NO: 203 - DNA, 17 bp, Salmo salar
taggcctatgtctagcc

SEQ ID NO: 204 - DNA, 18 bp, Salmo salar
gatgttagctatggctga

SEQ ID NO: 205 - DNA, 16 bp, Salmo salar
tacttctacttctcac

SEQ ID NO: 206 - DNA, 20 bp, Salmo salar
ctcatccgtaacattcacgc

SEQ ID NO: 207 - DNA, 16 bp, Capra hircus
tattcatacatatcgg

SEQ ID NO: 208 - DNA, 19 bp, Oryctolagus cuniculus
taggcctgtgccttataat

SEQ ID NO: 209 - DNA, 16 bp, Oryctolagus cuniculus
attcaaattttcactg

SEQ ID NO: 210 - DNA, 18 bp, Oryctolagus cuniculus
tctctactaggcctgtgc

SEQ ID NO: 211 - DNA, 21 bp, Oryctolagus cuniculus
tcaaattttcactggcctatt

SEQ ID NO: 212 - DNA, 17 bp, Oryctolagus cuniculus
tgccttataattcaaat

SEQ ID NO: 213 - DNA, 25 bp, Rattus norvegicus
acactacacgtctgataccataaca

SEQ ID NO: 214 - DNA, 17 bp, Rattus norvegicus
ctatttgcagtcatagc

SEQ ID NO: 215 - DNA, 17 bp, Rattus norvegicus
ggatcctacactttcct

SEQ ID NO: 216 - DNA, 22 bp, Rattus norvegicus
atgcctcatagtacaaatcctc

SEQ ID NO: 217 - DNA, 21 bp, Rattus norvegicus
aaacattgggatcatcctact

SEQ ID NO: 218 - DNA, 17 bp, Rattus norvegicus
ttcctccatgtgggacg

SEQ ID NO: 219 - DNA, 16 bp, Rattus norvegicus
gtatgcctcatagtac

SEQ ID NO: 220 - DNA, 19 bp, Salvelinus alpinus
tcatccggaatatccacgc

SEQ ID NO: 221 - DNA, 22 bp, Salvelinus alpinus
tggagtagtattactacttcta

SEQ ID NO: 222 - DNA, 23 bp, Salvelinus alpinus
ggcctatgtttggccacccaaat

SEQ ID NO: 223 - DNA, 23 bp, Salvelinus alpinus
tacttctaactataatgactgcc

SEQ ID NO: 224 - DNA, 16 bp, Salvelinus alpinus
ttggttcactcttagg

SEQ ID NO: 225 - DNA, 18 bp, Salvelinus alpinus
ttttcctctgtgtgccat

SEQ ID NO: 226 - DNA, 21 bp, Salvelinus alpinus
cctctgtgtgccatatctgcc

SEQ ID NO: 227 - DNA, 16 bp, Salvelinus fontinalis
tattattacttctcac

SEQ ID NO: 228 - DNA, 25 bp, Salvelinus fontinalis
tattggggtagtattattacttctc

SEQ ID NO: 229 - DNA, 19 bp, Salvelinus fontinalis
tctgtatgccacatttgtc

SEQ ID NO: 230 - DNA, 20 bp, Salvelinus fontinalis
ctcactataatgacagcttt

SEQ ID NO: 231 - DNA, 23 bp, Salvelinus fontinalis
tccgatatttcgacagctttttc

SEQ ID NO: 232 - DNA, 20 bp, Salvelinus fontinalis
atttatatgcatatcgcccg

SEQ ID NO: 233 - DNA, 26 bp, primer sequence CDL
ccatccaacatctcagcatgatgaaa

SEQ ID NO: 234 - DNA, 58 bp, primer sequence CBHT7
gaaattaatacgactcactatagggagaccacacccctcagaatgatatttgtcctca

SEQ ID NO: 235 - DNA, 14 bp, Bos taurus
gacacaacaacagc

SEQ ID NO: 236 - DNA, 14 bp, Gallus gallus
tccctagccttctc

SEQ ID NO: 237 - DNA, 14 bp, Gallus gallus
acacttgccggaac

SEQ ID NO: 238 - DNA, 14 bp, Bos taurus
atagccacagcatt

SEQ ID NO: 239 - DNA, 14 bp, Gadus morhua
ataataacctcttt

SEQ ID NO: 240 - DNA, 20 bp, primer sequence CBL 20
gacctcccagccccatcaaa

SEQ ID NO: 241 - DNA, 53 bp, primer sequence CBHT7 20
gaaattaatacgactcactatagggagaccacacagaatgatatttgtcctca

SEQ ID NO: 242 - DNA, 23 bp, Anguilla rostrata
tgcctataccttcacattgcccg

SEQ ID NO: 243 - DNA, 17 bp, Auxis thazard
attggcgtagttcttct

SEQ ID NO: 244 - DNA, 17 bp, Euthynnus alletteratus
ggcctgttcctcgcaat

SEQ ID NO: 245 - DNA, 19 bp, Euthynnus alletteratus
tttgcatttactcacacat

SEQ ID NO: 246 - DNA, 32 bp, Euthynnus alletteratus
aacattggtgtagtacttctactcctagtaat

SEQ ID NO: 247 - DNA, 25 bp, Euthynnus alletteratus
acttctactcctagtaatgataacc

SEQ ID NO: 248 - DNA, 17 bp, Gadus ogac  Gadus macrocephallus
catgctaacggtgcctc

SEQ ID NO: 249 - DNA, 26 bp, Gadus ogac  Gadus macrocephalus
tttttatttgtctctatatacatatt

SEQ ID NO: 250 - DNA, 30 bp, Gadus ogac  Gadus macrocephalus
tatttgtctctatatacatattgcccgagg

SEQ ID NO: 251 - DNA, 17 bp, Rangifer tarandus
tcctctgttactcacat

SEQ ID NO: 252 - DNA, 17 bp, Rangifer tarandus
cgagacgtcaattatgg

SEQ ID NO: 253 - DNA, 25 bp, Rangifer tarandus
gatcctcttatttacagtaatagct

SEQ ID NO: 254 - DNA, 34 bp, Rangifer tarandus
aatattggagtgatcctcttatttacagtaatag

SEQ ID NO: 255 - DNA, 29 bp, Salmo trutta  Salmo trutta fario
aatatcggagtcgtactgctacttctcac

SEQ ID NO: 256 - DNA, 17 bp, Salmo salar
taggcctatgtctagcc

SEQ ID NO: 257 - DNA, 19 bp, Salmo salar
gatgttagctatggctgac

SEQ ID NO: 258 - DNA, 20 bp, Salmo salar
ctcatccgtaacattcacgc

SEQ ID NO: 259 - DNA, 22 bp, Salmo salar
gagttgtacttctacttctcac

SEQ ID NO: 260 - DNA, 26 bp, Salmo salar
tttattatggttcctatctatataaa

SEQ ID NO: 261 - DNA, 23 bp, Thunnus thynnus
cttatttctcagatccttacagg

SEQ ID NO: 262 - DNA, 15 bp, Bos taurus
ctaatcctacaaatc

SEQ ID NO: 263 - DNA, 15 bp, Bos taurus
agcttcaatgttttt

SEQ ID NO: 264 - DNA, 15 bp, Gallus gallus
cggcctactactagc

SEQ ID NO: 265 - DNA, 15 bp, Gallus gallus
cacatccctagcctt

SEQ ID NO: 266 - DNA, 15 bp, Gallus gallus
gcccacacttgccgg

SEQ ID NO: 267 - DNA, 15 bp, Gallus gallus
ttgccggaacgtaca

SEQ ID NO: 268 - DNA, 15 bp, Gallus gallus
gaacgtacaatacgg

SEQ ID NO: 269 - DNA, 15 bp, Gallus gallus
tgaaacacaggagta

SEQ ID NO: 270 - DNA, 15 bp, Gadus morhua
tcagacatcgagaca

SEQ ID NO: 271 - DNA, 15 bp, Gadus morhua
gtaataataacctct

SEQ ID NO: 272 - DNA, 18 bp, primer
  misc_feature (4): n is I
agangcnccgtttgcgtg

SEQ ID NO: 273 - DNA, 20 bp, primer
  misc_feature (16): n is I
ttcttctttatctgtntcta

SEQ ID NO: 274 - DNA, 15 bp, primer
  misc_feature (4): n is I
rtcncgrcaratgtg

SEQ ID NO: 275 - DNA, 23 bp, primer
  misc_feature (3): n is I
gtnaaytwyggntgactnatccg

SEQ ID NO: 276 - DNA, 20 bp, primer
cagaatgatatttgtcctca

(19) United States
(12) Patent Application Publication
(10) Pub. No.: US 2005/0069890 A1
(43) Pub. Date: Mar. 31, 2005

(54) Method for the detection and/or identification of the original animal species in animal matter contained in a sample

(76) Inventors: Claude Mabilat, Saint Germain Au Mont D'or (FR); Sabine Desvarenne, Decines Charpieu (FR); Odile Babola, Decines Charpieu (FR); Bruno Lacroix, Saint Genis Laval (FR); Natalia Bello Pigem, Cambrils (ES)

Correspondence Address: OLIFF & BERRIDGE, PLC, P.O. BOX 19928, ALEXANDRIA, VA, 22320, US

(21) Appl. No.: 10/500,646

(22) PCT Filed: Jan. 10, 2003
(86) PCT No.: PCT/FR03/00078

(30) Foreign Application Priority Data
Jan. 10, 2002 (FR) 02/00265

(51) Int. Cl. C12Q 1/68

(52) U.S. Cl. 435/6

(57) ABSTRACT

Disclosed is a method for detecting or identifying the original animal species in a sample likely to contain an ingredient obtained at least from said species. The inventive method is characterized by the following steps: a) a nuclear fraction is obtained from said sample; b) at least one reactant which is specific to the animal species is provided and selected among a group formed by: - the reference sequences SEQ ID numbers 1 to 232, 242 to 261; - the sequences complementing each of the sequences SED ID numbers 1 to 232, 242 to 261, respectively; - the sequences homologous to each of the sequences SEQ ID numbers 1 to 232, 242 to 261 and sequences complementing each of the sequences SED ID numbers 1 to 232, 242 to 261, respectively; c) the nuclear fraction and said reactant are reacted with each other; and d) any signal or information resulting from the specific reaction between said reactant and the nuclear fraction is detected, whereby it can be established if said sample contains said original animal species.

The present invention relates to the field of the determination of an animal species, hereinafter referred to as original animal species, in a sample liable to contain an ingredient, itself obtained from at least said species. The products on which the determination according to the present invention is carried out are, for example, foods or foodstuffs intended for humans or animals, cosmetic products and, in general, products liable to contain ingredients of animal origin or, on the contrary, products in which these extracts are prohibited.

For example, identifying the animal species present in foods may be necessary in many fields of activity. A first reason is to combat fraudulent foods in which certain animal species are substituted with less expensive species, such as replacing hare with rabbit. A second reason is public health, for instance especially during the bovine spongiform encephalitis, or BSE, epidemic, a disease due to the use of animal meat meals of bovine origin for bovine feed. A third reason is religious in nature, in order to verify, for example, the absence of pork in foods. A fourth reason is legislative in nature, in particular in verifying the absence of protected species in foods.

Three main identification approaches are currently described in the literature; these methods are based on a tissue or microscopic analysis, on a protein analysis and/or on a genetic analysis.

The tissue analysis thus consists in determining the presence of bone fragments in samples of meals intended for animal feed. This technique, described in particular in the article by Michard, Revue de l'alimentation animale [Animal feed review], vol. 508, pp 43-48, 1997, although sensitive, is laborious and is based on an expert's interpretation. It is therefore difficult to compare from one laboratory to another. In addition, by nature, it cannot detect the addition of soft tissues, such as offal, serum, blood tissues, gelatin.

Among the protein analyses used, three groups of methods for identifying animal species present in a given sample are mainly distinguished in the literature.

The first group of methods comprises protein electrophoresis techniques, which consist in detecting the soluble target proteins by specific enzymatic staining. The diagnosis is obtained after polyacrylamide gel electrophoresis, for example. However, this technique can only be carried out with fresh or frozen, unprocessed tissues, since cooking the food for a period of time is an example of processing liable to alter the proteins. This technique cannot therefore be applied to the detection of animal species present in plant meals, which undergo cooking phases during their manufacture.

The second group of methods is based on immunological techniques, using antibodies directed against soluble target proteins. The “Ouchterlony”, or double immuno-diffusion, technique, a method used to differentiate antigens in a mixture, can be used. However, this technique has the major disadvantage of involving cross reactions with the epitopes of other species. The use of ELISA (enzyme-linked immunosorbent assay) techniques allows better discrimination between the species, and these techniques can be applied to cooked meat when antibodies directed against thermoresistant epitopes are used. However, problems of specificity are again observed. By way of indication, polyclonal antibodies directed against thermoresistant epitopes from chicken are not sufficiently specific to determine whether chicken meat or turkey meat is involved.

The third group of methods comprises the chromatographic (HPLC) techniques used to characterize soluble muscle proteins. However, these techniques remain technically laborious and expensive, and can only be applied to fresh or recently frozen tissues.

The disadvantages of these three methods are mainly due to their dependence on the characterization of proteins which are thermosensitive, which denature when the foods are cooked for a period of time and which lose their biological activity after the animal's death, and the presence of which often depends on the cell type that is examined.

It is thus preferable to directly analyze the DNA, rather than the proteins, of the sample, in order to identify the original animal species which is or are present in a given sample, the DNA being identical in all the cell types of the same animal and stable by comparison with the proteins. A third approach therefore consists in analyzing the DNA present in the sample. Only recently have methods based in particular on the use of restriction enzymes or of genetic markers thus been found in the literature, these methods having the advantage of being able to be applied to processed products, in particular after thermal treatment.

The nucleic acid determination may make use of restriction enzymes, or the technique referred to as RFLP (Restriction Fragment Length Polymorphism, see in particular Meyer et al., Journal of AOAC International, vol 78 No. 6, pp 1542-1551, 1995). The restriction enzymes cleave the DNA, extracted beforehand from the sample to be analyzed, at precise sites in the macromolecule. It then suffices to compare, by simple electrophoresis, the fragments obtained with those of control samples representative of the species to be identified. However, the analysis of the results obtained by this technique is very tricky, in particular when several animal species are present in the sample.

The nucleic acid determination can also consist in sequencing a ubiquitous marker, such as mitochondrial DNA cytochrome B. Mitochondrial DNA is a known target for this type of analysis since each mitochondrion contains from one to ten mitochondrial DNA molecules, and each cell contains from a few tens to a few thousand mitochondria, which makes it possible to work on a very small amount of sample. Thus, Bartlett & Davidson (Biotechniques, vol. 12, No. 3, 1992) describe a method called FINS (Forensically Informative Nucleotide Sequencing). This method consists in i) isolating the DNA present in a biological sample, ii) amplifying this DNA by PCR using primers specific for the mitochondrial cytochrome B gene, the primers being chosen in the portion of the gene which is highly conserved during evolution, and iii) sequencing the amplified DNA segment. The sequence is then used for a phylogenetic analysis by means of a database, allowing identification of the animal species initially present in the sample. While this method has the advantage of being rapid and usable on any type of foods (fresh, frozen, processed, etc.), it nevertheless has the major disadvantage of not enabling the analysis of mixtures of species, from mixtures of amplified sequences derived from the same ubiquitous polymorphic marker, and thus remains reserved for homogeneous starting materials.

The analysis can also consist in amplifying a marker specific for a given species. Thus, Lahiff et al. (Molecular and Cellular Probes, vol. 15, pp 27-35, 2001) describe the identification of an ovine, bovine or avian species present in a sample using, by PCR, particular primers specific to each species. A method developed by S. Colgan et al. was also described in 2001 (FOOD Research International, 2001, vol 34, No. 5, 401-414), for detecting 4 species in a mixture using specific primers by PCR. While this method makes it possible to specifically and rapidly identify such and such a species, it cannot be applied simultaneously to the detection of several species. Successive PCRs are then necessary if the detection of several species is desired. The detection of six animal species using a multiplex PCR (Matsunaga et al. 1999 Meat Sciences, (1999), 145-148) and (Matsunaga T., et al., Nippon Shokuhin KogakuKaishi, (1999) vol 46. No. 3, 187-194) is thus found in the prior art. However, this technique remains tricky and difficult to apply and, in practice, involves prior knowledge of the species sought. This technique cannot, however, be applied blind, i.e. without prior knowledge of the species likely to be present in the sample. It does not make it possible to have quantitative results because of the difficulties due to the multiplex amplification and the possibilities of mismatches. In addition, this technique requires a large number of specific primers if the intention is to test a large number of species, which is relatively impossible to realize in practice due to problems of sensitivity and specificity. Finally, if a species is not represented in the set of primers but is nevertheless present in the sample to be analyzed, the result will be distorted.

The techniques described above make it possible to determine, without prior knowledge, the species present when the sample comprises only one species, and they make it possible to detect several species when there is prior knowledge of the species brought together, but none of the techniques mentioned above allows a determination in the presence of a mixture of several species without prior knowledge of said species brought together. In addition, most of the techniques described above, when several species are present, do not allow a reliable determination when the proportions of the various species are very different in the sample.

There is therefore a great need for a technique which, while remaining generic, can detect one or more species, even present in large number in the same sample to be analyzed or in very small amount, and without prior knowledge of the species present.

In fact, while, in a product, the unwanted species must be present in amounts greater than 5% or even 1% according to the legislation, relative to the species normally present in order for there to be fraudulent practice, which eases the required performance levels for the molecular diagnostic test, it is quite different in the case of products in which the presence of products of animal origin is prohibited. For example, in the case of meals used in France for animal feed since Jan. 1, 2001, traces of content of product of animal origin are sought, and the technical constraint is considerable in terms of sensitivity of the method since most of the material is of plant origin and the addition of animal material ranges between 0.1 and 5% weight/weight.

A need therefore exists for a determining tool which allows the qualitative and/or quantitative identification or detection of animal species, blind, i.e. without a priori regarding the identity of the species sought, which can be used simply, while remaining specific, reliable and accurate, and which can be used in a medium possibly containing ingredients obtained from several animal species.

The problem to be solved is of considerable complexity. The determination must be possible blind, i.e. the sample may or may not contain ingredients obtained from one or more animal species and these original species are unknown. If the sample contains ingredients obtained from animal species, the original species must be determined and may be related, and it must be possible to make the determination by carrying out just one analysis, with a single reagent and a single amplification step, without a prior step for predetermining, for example, the group of species or without using batteries of tests making it possible, for example, to classify the reagents by genera or species so as to avoid, for example, cross reactions.

To this effect, the applicant has discovered a set of sequences consisting of the group comprising the sequences SEQ ID Nos 1 to 232, 242 to 261, the sequences respectively complementary thereto, and any homologous sequences, comprising at least 5 contiguous monomers included in any one of said sequences and exhibiting at least 70% identity with said any sequence, which make it possible, using “molecular biology” analytical methods, to determine at least one original animal species in a sample liable to contain an ingredient obtained from at least said species.

Before disclosing the invention, various terms used in the description and the claims are defined hereinafter.
A “determination” is understood to be the identification or the quantitative and/or qualitative detection or analysis of an animal species.
An “animal species” is understood to be the simplest category used in the classification of living species or taxonomy. Living species are classified in categories called taxa; the most important taxa are the kingdom (plant or animal), the phyllum or division, the class, the order, the family, the genus and the species. Birds, fish and mammals are classes of vertebrate animals.
The term “original animal species” is understood to mean the animal species of the animal whose tissues, whatever they are, were used as starting material for preparing the ingredient(s) of the sample of the product subjected to the determination according to the present invention.
A “molecular biology method” is a method based on the enzymatic amplification of nucleic acid (DNA and/or RNA) targets in vitro and the use of oligonucleotide probes.
A “sample” is any part obtained directly or indirectly from a starting product, matter or material, itself liable to contain at least one ingredient obtained from at least one “original” animal species. As a consequence of this definition, the sample to be determined in accordance with the present invention is liable to contain said ingredient of animal origin, based on which the animal species which has or have made up or constituted the starting product, matter or material is or are identified or detected. For the purpose of the present invention, the starting product can be a biological material, a food or foodstuff, for example based on meat or fish, a cosmetic product, etc.
The term “lysis step” is understood to mean a step capable of releasing the nucleic acids contained in the protein and/or lipid envelopes of the microorganisms (such as cell debris which disturbs the subsequent reactions). By way of example, use may be made of the lysis methods as described in the applicant's patent applications:
   WO-A-00/05338 regarding mixed magnetic and mechanical lysis,
   WO-A-99/53304 regarding electrical lysis, and
   WO-A-99/15321 regarding mechanical lysis.

Those skilled in the art may use other well-known lysis methods, such as thermal or osmotic shocks or chemical lyses with chaotropic agents such as guanidium salts (U.S. Pat. No. 5,234,809).
The term “purification” is understood to mean separation between the nucleic acids and the other cell components released in the lysis step. This step generally makes it possible to concentrate the nucleic acids. By way of example, it is possible to use magnetic particles optionally coated with oligonucleotides, by adsorption or covalence (on this subject, see patents U.S. Pat. No. 4,672,040 and U.S. Pat. No. 5,750,338), and thus to purify the nucleic acids which are attached to these magnetic particles, by means of a washing step. This nucleic acid purification step is particularly advantageous if it is desired to subsequently amplify said nucleic acids. A particularly advantageous embodiment of these magnetic particles is described in the patent applications filed by the applicant under the following references: WO-A-97/45202 and WO-A-99/35500.

In the latter of these patent applications, the particles are thermosensitive magnetic particles which each have a magnetic core covered with an intermediate layer. The intermediate layer is itself covered with an outer layer based on a polymer capable of interacting with at least one biological molecule, for example nucleic acid; the outer polymer is thermosensitive and has a predetermined lower critical solution temperature (LCST) of between 10 and 100° C., and preferably between 20 and 60° C. This outer layer is synthesized from cationic monomers which generate a polymer having the ability to bind nucleic acids. This intermediate layer isolates the core's magnetic forces in order to avoid problems of inhibition of the techniques for amplifying these nucleic acids.

Another advantageous example of a method for purifying nucleic acids is the use of silica, either in the form of a column (Qiagen kits, for example), or in the form of inert particles [Boom R. et al., J. Clin. Microbiol., 1990, No. 28(3), p. 495-503] or magnetic particles (Merck: MagPrep® Silica, Promega: MagneSil™ Paramagnetic particles). Other very widely used methods are based on ion exchange resins in a column (Qiagen kits, for example) or in a paramagnetic particulate format (Whatman: DEAE-Magarose) [Levison P R et al., J. Chromatography, 1998, p. 337-344]. Another method which is very relevant but not exclusive for the invention is that of adsorption onto a metal oxide support (Xtrana: Xtra-Bind™ matrix).
A “sequence”, or a “nucleotide fragment”, or an oligonucleotide or a polynucleotide, is a chain of nucleotide units assembled together via phosphoester bonds, characterized by the informational sequence of the natural nucleic acids capable of hybridizing with a nucleotide fragment, it being possible for the chain to contain monomers having different structures and to be obtained from a natural nucleic acid molecule and/or by genetic recombination and/or by chemical synthesis.

A “unit” is derived from a monomer which may be a natural nucleotide of nucleic acid, of which the constituent elements are a sugar, a phosphate group and a nitrogenous base; in DNA, the sugar is 2-deoxyribose, and in RNA, the sugar is ribose; depending on whether it is a question of DNA or RNA, the nitrogenous base is chosen from adenine, guanine, uracil, cytosine and thymine; or alternatively the monomer is a nucleotide which has been modified in at least one of the three constituent elements; by way of example, the modification can affect either the bases, with modified bases such as inosine, 5-methyldeoxycytidine, deoxyuridine, 5-dimethylaminodeoxyuridine, 2,6-diaminopurine, 5-bromodeoxyuridine or any other modified base capable of hydridization, or the sugar, for example the replacement of at least one deoxyribose with a polyamide (P. E. Nielsen et al., Science, 254, 1497-1500 (1991)), or alternatively the phosphate group, for example replacement thereof with esters chosen in particular from diphosphates, alkyl- and arylphosphonates and phosphorothioates.
The term “informational sequence” is understood to mean any ordered series of units of nucleotide type, the chemical nature of which and the order of which in a reference direction constitute an item of information of the same quality as that of the natural nucleic acids.
The term “hybridization” is understood to mean the process during which, under suitable conditions, two nucleotide fragments having sufficiently complementary sequences are capable of forming a double strand with stable and specific hydrogen bonds. A nucleotide fragment “capable of hybridizing” with a polynucleotide is a fragment which can hybridize with said polynucleotide under hybridization conditions which can be determined in a known manner in each case. The hybridization conditions are determined by means of the stringency, i.e. the severity of the operating conditions. The higher the stringency at which the hybridization is carried out, the more specific the hybridization is. The stringency is defined in particular according to the base composition of a probe/target duplex, and also by means of the degree of mismatching between two nucleic acids.

The “stringency” can also depend on the parameters of the reaction, such as the concentration and the type of ion species present in the hybridization solution, the nature and the concentration of denaturing agents and/or the hybridization temperature. The stringency of the conditions under which a hybridization reaction should be carried out will depend mainly on the target probes used. All these data are well known and the appropriate conditions can be determined by those skilled in the art.

In general, depending on the length of the target probes used, the temperature for the hybridization reaction is between approximately 20 and 70° C., in particular between 35 and 65° C., in a saline solution at a concentration of approximately 0.5 to 1 M.
A “probe” comprises a nucleotide fragment comprising from 5 to 100 monomers, in particular from 6 to 35 monomers, possessing a hybridization specificity under given conditions so as to form a hybridization complex with a nucleotide fragment having, in the present case, a nucleotide sequence included, for example, in a ribosomal RNA, the DNA obtained by reverse transcription of said ribosomal RNA, and the DNA (referred to here as ribosomal DNA or rDNA) for which said ribosomal RNA is the transcription product; a probe can be a capture probe or a detection probe.
A “capture probe” is immobilized or can be immobilized on a solid support by any suitable means, i.e. directly or indirectly, for example by covalence or adsorption.
A “detection probe” can be labeled by means of a label chosen from radioactive isotopes, enzymes (in particular a peroxidase, an alkaline phosphatase, or an enzyme capable of hydrolyzing a chromogenic, fluorigenic or luminescent substrate), chemical chromophore compounds, chromogenic, fluorigenic or luminescent compounds, nucleotide base analogs, and ligands such as biotin.
A “primer” comprises a nucleotide fragment comprising from 5 to 100 nucleotide units and possessing a hybridization specificity under given conditions for the initiation of an enzymatic polymerization, for example in an amplification technique, in a sequencing process, in a reverse transcription method, etc.
“The homology” characterizes the degree of identity of two compared nucleotide fragments, for which the criteria selected for the present invention are defined below.

The probes and primers according to the invention are chosen from:
(a) the sequences identified in the sequence listing attached in the appendix to the description,
(b) the sequences complementary to each of the sequences identified in the sequence listing attached in the appendix to the description, the complementarity meaning any sequence capable of hybridizing, at a temperature of between 20 and 70° C., and preferably between 35 and 65° C., in saline solution at a concentration of approximately 0.5 to 1 M, and preferably 0.8 to 1 M, with any one of the sequences identified in the sequence listing attached in the appendix to the description,
(c) the sequences homologous to each of the sequences identified in the sequence listing attached in the appendix to the description, and of the sequences complementary to each of the sequences identified in the sequence listing attached in the appendix to the description, respectively, the homology meaning any sequence, for example fragment, comprising a series of at least 5 contiguous nucleotides included in any one of said sequences, and exhibiting at least 70% identity with said any sequence; by way of example, a fragment (c) comprises 10 nucleotides, among which 5 contiguous nucleotides belong to a sequence (a) and at least two nucleotides of the remaining 5 nucleotides are identical, respectively, to the two corresponding nucleotides in the reference sequence, after alignment.
   The term “identifying sequence” denotes any sequence or any fragment as defined above, which can serve as a detection probe and/or capture probe.
   The term “detection” is understood to mean either a direct detection by means of a physical method, or a method of detection using a label.

Many detection methods exist for detecting nucleic acids [see, for example, Kricka et al., Clinical Chemistry, 1999, No. 45(4), p. 453-458 or Keller G. H. et al., DNA Probes, 2nd Ed., Stockton Press, 1993, sections 5 and 6, p. 173-249].

The term “label” is understood to mean a tracer capable of engendering a signal. A nonlimiting list of these tracers comprises the enzymes which produce a signal that can be detected, for example, by colorimetry, fluorescence or luminescence, such as horseradish peroxidase, alkaline phosphatase, beta-galactosidase, glucose-6-phosphate dehydrogenase; chromophors such as fluorescent, luminescent or dye compounds; electron dense groups which can be detected by electron microscopy or by means of their electrical properties such as conductivity, by amperometry or voltametry methods, or by impedance measurements; groups which can be detected by optical methods such as diffraction, surface plasmon resonance or contact angle variation or by physical methods such as atomic force spectroscopy, tunnel effect, etc.; radioactive molecules such as ³²P, ³⁵S or ¹²⁵I.

Thus, the polynucleotide can be labeled during the enzymatic amplification step, for example by using a labeled triphosphate nucleotide for the amplification reaction. The labeled nucleotide will be a deoxyribonucleotide in amplification systems generating a DNA, such as PCR, or a ribonucleotide in amplification techniques generating an RNA, such as the TMA or NASBA techniques.

The polynucleotide can also be labeled after the amplification step, for example by hybridizing a labeled probe according to the sandwich hybridization technique described in document WO-A-91/19812.

Another particularly preferred method for labeling nucleic acids is described in the applicant's application FR-A-2 780 059. Another preferred method of detection uses the 5′-3′ exonuclease activity of a polymerase, as described by Holland P. M., PNAS (1991) p 7276-7280.

Signal amplification systems can be used as described in document WO-A-95/08000 and, in this case, the preliminary enzymatic amplification reaction may not be necessary.
The term “enzymatic amplification” is understood to mean a process generating multiple copies of a particular nucleotide fragment using specific primers by means of the action of at least one enzyme. Thus, for nucleic acid amplification, there exists, inter alia, the following techniques:
PCR (Polymerase Chain Reaction), as described in patents U.S. Pat. No. 4,683,195, U.S. Pat. No. 4,683,202 and U.S. Pat. No. 4,800,159,
LCR (Ligase Chain Reaction), disclosed, for example, in patent application EP-A-0 201 184,
RCR (Repair Chain Reaction), described in patent application WO-A-90/01069,
3SR (Self Sustained Sequence Replication) with patent application WO-A-90/06995,
NASBA (Nucleic Acid Sequence-Based Amplification) with patent application WO-A-91/02818, and
TMA (Transcription Mediated Amplification) with patent U.S. Pat. No. 5,399,491.

The term “amplicons” is then used to denote the polynucleotides generated by means of an enzymatic amplification technique.
The term “solid support” as used here includes all the materials on which a nucleic acid can be immobilized. Synthetic materials or natural materials, optionally chemically modified, can be used as a solid support, in particular polysaccharides such as cellulose-based materials, for example paper, cellulose derivatives such as cellulose acetate and nitrocellulose or dextran, polymers, copolymers, in particular based on monomers of the styrene type, natural fibers such as cotton, and synthetic fibers such as nylon; inorganic materials such as silica, quartz, glasses, ceramics; latices; magnetic particles; metal derivatives, gels, etc. The solid support can be in the form of a microtitration plate, of a membrane as described in application WO-A-94/12670, of a particle or of a biochip.
The term “biochip” is understood to mean a solid support which is small in size and to which is attached a multitude of capture probes at predetermined positions.

By way of illustration, examples of these biochips are given in the publications by [G. Ramsay, Nature Biotechnology, 1998, No. 16, p. 40-44; F. Ginot, Human Mutation, 1997, No. 10, p. 1-10; J. Cheng et al., Molecular diagnosis, 1996, No. 1(3), p. 183-200; T. Livache et al., Nucleic Acids Research, 1994, No. 22(15), p. 2915-2921; J. Cheng et al., Nature Biotechnology, 1998, No. 16, p. 541-546] on in patents U.S. Pat. No. 4,981,783, U.S. Pat. No. 5,700,637, U.S. Pat. No. 5,445,934, U.S. Pat. No. 5,744,305 and U.S. Pat. No. 5,807,522.

The main characteristic of the solid support should be to conserve the characteristics of hybridization of the capture probes to the nucleic acids while at the same time generating a minimum background noise for the detection method. An advantage of biochips is that they simplify the use of many capture probes, thus allowing multiple detection of the species to be detected.

The invention described hereinafter makes it possible to solve the problems posed by the methods described above, equally in terms of sensitivity, specificity, multidetection capacity and identification, while at the same time being rapid and easy to implement.

The invention relates to a method for determining an original animal species in a sample liable to contain an ingredient obtained from at least said species, characterized in that:
a) a nucleic acid fraction obtained from said sample is provided,
b) at least one reagent specific for the animal species is provided, chosen from the group consisting of
   the reference sequences SEQ ID Nos 1 to 232, and Nos 242 to 261,
   the sequences complementary to each of the sequences SEQ ID Nos 1 to 232, and Nos 242 to 261, respectively, the complementarity meaning any sequence capable of hybridizing, at a temperature of between 20 and 70° C., and preferably between 35 and 65° C., in saline solution at a concentration of approximately 0.5 to 1 M, and preferably 0.8 to 1 M, with any one of the sequences SEQ ID Nos 1 to 232, and Nos 242 to 261,
   the sequences homologous to each of the sequences SEQ ID Nos 1 to 232, and Nos 242 to 261 and of the sequences complementary to each of the sequences SEQ ID Nos 1 to 232, and Nos 242 to 261, respectively, the homology meaning any sequence, for example fragment, comprising a series of at least 5 contiguous nucleotides included in any one of said sequences, and exhibiting at least 70% identity with said any sequence,
c) the nucleic acid fraction and said reagent are brought into contact, and
d) any signal or item of information resulting from the specific reaction between said reagent and the nucleic acid fraction, characterizing the presence in said sample of said original animal species, is determined by means of detection.

It also relates to a method as described above, characterized in that a set comprising a multiplicity of said reagents specific for the same original species and/or for respectively different original animal species is provided; and a multiplicity of signals or items of information characterizing the presence in said sample of the same original animal species or of several respectively different original animal species is determined.

It also relates to any nucleotide sequence characterized in that it is chosen from the group consisting of:
a) the reference sequences SEQ ID Nos 1 to 232, and Nos 242 to 261,
b) the sequences complementary to each of the sequences SEQ ID Nos 1 to 232, and Nos 242 to 261, respectively, the complementarity meaning any sequence capable of hybridizing, at a temperature of between 20 and 70° C., and preferably between 35 and 65° C., in saline solution at a concentration of approximately 0.5 to 1 M, and preferably 0.8 to 1 M, with any one of the sequences SEQ ID Nos 1 to 232, and Nos 242 to 261,
c) the sequences homologous to each of the sequences SEQ ID Nos 1 to 232, and Nos 242 to 261, and of the sequences according to b), respectively, the homology meaning any sequence, for example fragment, comprising a series of at least 5 contiguous nucleotides included in any one of said sequences, and exhibiting at least 70% identity with said any sequence.

It also relates to the use of a sequence defined above, for determining at least one original animal species in a sample liable to contain an ingredient obtained from at least said animal species.

The invention relates to a method for determining an original animal species in a sample liable to contain an ingredient obtained from at least said species, characterized in that it allows said determination in a sample containing at least one other ingredient obtained from another animal species and without prior knowledge of the species brought together, and in that:
a) a nucleic acid fraction obtained from said sample is provided,
b) at least one reagent specific for the animal species is provided, chosen from the group consisting of:
   the reference sequences SEQ ID Nos 1 to 232, and Nos 242 to 261,
   the sequences complementary to each of the sequences SEQ ID Nos 1 to 232, and Nos 242 to 261, respectively, the complementarity meaning any sequence capable of hybridizing, at a temperature of between 20 and 70° C., and preferably between 35 and 65° C., in saline solution at a concentration of approximately 0.5 to 1 M, and preferably 0.8 to 1 M, with any one of the sequences SEQ ID Nos 1 to 232, and Nos 242 to 261,
   the sequences homologous to each of the sequences SEQ ID Nos 1 to 232, and Nos 242 to 261, and of the sequences complementary to each of the sequences SEQ ID Nos 1 to 232, and Nos 242 to 261, respectively, the homology meaning any sequence, for example fragment, comprising a series of at least 5 contiguous nucleotides included in any one of said sequences, and exhibiting at least 70% identity with said any sequence,
c) the nucleic acid fraction and said reagent are brought into contact, and
d) any signal or item of information resulting from the specific reaction between said reagent and the nucleic acid fraction, characterizing the presence in said sample of said original animal species, is determined by means of detection.

The invention can also be a probe for determining at least one original animal species, comprising at least one identifying nucleotide sequence defined above.

It also relates to a primer for the specific amplification of a nucleic acid from an original animal species, comprising at least one identifying nucleotide sequence defined above.

Another embodiment of the invention is a reagent for determining at least one original animal species, comprising a solid support, which may or may not be divided up, to which a nucleotide sequence defined above is attached.

According to the invention, the nucleotide sequences or their fragments can be attached to a solid support and can constitute a biochip which makes it possible to determine the multiplicity of signals or items of information.

The method according to the invention can be carried out manually, semi-automatically or automatically, allowing the use of a means for determining the original animal species in animal matter contained in a sample.

This invention also relates to a method of detection using in particular the biochip technique. This method of detection is specific for the species being sought by virtue of the use of sequences, referred to as identifying sequences for each species, as a probe. The rapidity, the sensitivity and the specificity of this method of detection make it possible to apply it equally to any medium. In particular, this method applies to any sample of a food product comprising animal matter, whatever its condition and the methods of manufacture and/or of production used, in particular the cooking, dehydration and/or storage techniques, and to any sample of a manufactured product liable to contain animal extracts, such as, for example, cosmetic products and/or pharmaceutical products comprising, for example, gelatins of animal origin.

This simultaneous single-step detection of multiple specific amplification products is possible by virtue of the use of a solid support, in particular in the form of a solid support which is small in size and to which is attached a multitude of capture probes at predetermined positions, or “biochip”, these capture probes consisting of a set of fragments of, or of all, nucleotide sequences specific for said identifying sequences for the species being sought.

These nucleotide sequences can also be used in all the known hybridization techniques, such as the “Dot-blot” techniques for depositing a spot onto a filter [Maniatis et al., Molecular Cloning, Cold Spring Harbor, 1982], the “Southern blot” techniques for transferring DNA [Southern E. M., J. Mol. Biol., 1975, 98, 503], the “Northern blot” techniques for transferring RNA, or the “Sandwich” techniques [Dunn A. R. et al., Cell, 1977, 12,23].

The present invention also relates to the determination of a group of species or class of animal species or taxon. These groups of species or classes or taxa consist, for example, of a class, such as the class of mammals, birds or fish, or even of subgroups of species such as a family of birds or of two subgroups combined, such as birds or mammals.

This identification is possible through the identification of nucleotide sequences, called signature sequences, characteristic of a class, of a group, of a subgroup or of a taxon, and corresponding to regions which have been conserved for all the individuals making up the group. Any signature sequence specific for a class of animals, used in the method according to the present invention, exhibits the characteristic according to which, firstly, it has a nucleic acid region which has been conserved for virtually all the animal species of the same taxonomic class and, secondly, it can be distinguished from other sequences corresponding to the same definition as above, under the usual conditions for determination, defined generically in the attached claims.

The invention also relates to a method for determining a group of original animal species in a sample liable to contain an ingredient obtained from at least one species belonging to said group of animal species under consideration, characterized in that:
a) a nucleic acid fraction obtained from said sample is provided,
b) the nucleotide sequence(s) characteristic of the group of animal species to be determined is (are) identified,
c) at least one reagent comprising a sequence identified in step b) is provided,
c) the nucleic acid fraction and said reagent are brought into contact, and
d) any signal or item of information resulting from the presence of one of the sequences defined above, characterizing the presence in said sample of a group of original animal species, is determined by means of detection.

For example, for detecting the presence of mammals, use will be made of:
1/ the signature sequence M1, corresponding to the sequence SEQ ID No. 235 GACACAACAA CAGC, positions 14685 to 14698 (genbank Bos taurus reference sequence; accession No. V00654). The CAA bases at positions 14689-14690-14691 (genbank Bos taurus reference sequence; accession No. V00654) are conserved for all the nucleic acid material corresponding to the predefined species making up the group that it is desired to investigate, in this case mammals. No more than 5 mutated positions are observed for the remainder of the cited signature for all the sequences making up the nucleic acid material of the group of chosen mammals. The presence of these three bases at the positions indicated above thus makes it possible to determine the presence of mammals in the sample;
2/ the signature sequence M2, corresponding to the sequence SEQ ID No. 262, positions 14634 to 14648 (genbank Bos taurus reference sequence; accession No. V00654). The T base at position 14641 (genbank Bos taurus reference sequence; accession No. V00654) is conserved for all the nucleic acid material corresponding to the predefined species making up the group that it is desired to investigate, in this case mammals. No more than 5 mutated positions are observed for the remainder of the cited signature for all the sequences making up the nucleic acid material of the group of chosen mammals. The presence of this base at the position indicated above thus makes it possible to determine the presence of mammals in the sample;
3/ the signature sequence M3, corresponding to the sequence SEQ ID No. 263, positions 14771 to 14785 (genbank Bos taurus reference sequence; accession No. V00654). The A base at position 14778 (genbank Bos taurus reference sequence; accession No. V00654) is conserved for all the nucleic acid material corresponding to the predefined species making up the group that it is desired to investigate, in this case mammals. No more than 5 mutated positions are observed for the remainder of the cited signature for all the sequences making up the nucleic acid material of the group of chosen mammals. The presence of this base at the position indicated above thus makes it possible to determine the presence of mammals in the sample.

Identification of the presence of birds is determined by means of the signatures:
1/ O1, corresponding to the sequence SEQ ID No. 236 TCCCTAGCCT TCTC, positions 15073 to 15086 (Gallus gallus reference sequence; genbank accession No. X52392). The CT bases (positions 15076-15077) are conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of these two bases at the positions indicated above thus makes it possible to determine the presence of birds in the sample.
2/ O2, corresponding to the sequence SEQ ID No. 237 ACACTTGCCG GAAC, positions 15098 to 15111 (Gallus gallus reference sequence; genbank accession No. X52392). The CT or CA bases (positions 15101-15102) are conserved for all the nucleic acid material corresponding to the predefined species making up the group that it is desired to investigate, in this case birds. No more than 4 mutated positions are observed for the remainder of the cited signature for all the sequences making up the nucleic acid material of the group of chosen birds. The presence of these two bases at the positions indicated above thus makes it possible to determine the presence of birds in the sample.
3/ O3, corresponding to the sequence SEQ ID No. 264, positions 15036 to 15050 (genbank Gallus gallus reference sequence; accession No. X52392). The C base at position 15043 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
4/ O4, corresponding to the sequence SEQ ID No. 265, positions 15069 to 15083 (genbank Gallus gallus reference sequence; accession No. X52392). The C base at position 15076 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
5/ O5, corresponding to the sequence SEQ ID No. 266, positions 15094 to 15108 (genbank Gallus gallus reference sequence; accession No. X52392). The C base at position 15101 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
6/ O6, corresponding to the sequence SEQ ID No. 267, positions 15102 to 15116 (genbank Gallus gallus reference sequence; accession No. X52392). The A base at position 15109 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
7/ O7, corresponding to the sequence SEQ ID No. 268, positions 15108 to 15122 (genbank Gallus gallus reference sequence; accession No. X52392). The C base at position 15115 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
8/ O8, corresponding to the sequence SEQ ID No. 269, positions 15232 to 15246 (genbank Gallus gallus reference sequence; accession No. X52392). The C base at position 15239 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.

Identification of the presence of mammals and of birds is determined by means of the signature V, corresponding to the sequence SEQ ID No. 238 ATAGCCACAGCATT, positions 14883 to 14896 (genbank Bos taurus reference sequence; accession No. V00654). The GC bases (at positions 14886 and 14887) are conserved for all the nucleic acid material corresponding to the predefined species making up the group that it is desired to investigate, in this case birds and mammals. No more than 4 mutated positions are observed for the remainder of the cited signature for all the sequences making up the nucleic acid material of the group of chosen birds and mammals. The presence of these two bases at the positions indicated above thus makes it possible to determine the presence of mammals and of birds in the sample.

Identification of the presence of fish is determined by means of:
1/ the signature P1, corresponding to the sequence SEQ ID No. 239 ATAATAACCTCTTT, positions 14713 to 14726 (Gadus morhua reference sequence; genbank accession No. X99772). The ATA or ATG bases (positions 14716-14717-14718) are conserved for all the nucleic acid material corresponding to the predefined species making up the group that it is desired to investigate, in this case fish. No more than 4 mutated positions are observed for the remainder of the cited signature for all the sequences making up the nucleic acid material of the group of chosen fish. The presence of these three bases at the positions indicated above thus makes it possible to determine the presence of fish in the sample;
2/ the signature sequence of P2, corresponding to the sequence SEQ ID No. 270, positions 14512 to 14526 (genbank Gadus morhua reference sequence; accession No. X99772). The T base at position 14519 (genbank Gadus morhua reference sequence; accession No. X99772) is conserved for all the nucleic acid material corresponding to the predefined species making up the group that it is desired to investigate, in this case fish. No more than 5 mutated positions are observed for the remainder of the cited signature for all the sequences making up the nucleic acid material of the group of chosen fish. The presence of this base at the position indicated above thus makes it possible to determine the presence of fish in the sample;
3/ the signature sequence P3, corresponding to the sequence SEQ ID No. 271, positions 14710 to 14724 (genbank Gadus morhua reference sequence; accession No. X99772). The T base at position 14717 (genbank Gadus morhua reference sequence; accession No. X99772) is conserved for all the nucleic acid material corresponding to the predefined species making up the group that it is desired to investigate, in this case fish. No more than 5 mutated positions are observed for the remainder of the cited signature for all the sequences making up the nucleic acid material of the group of chosen fish. The presence of this base at the position indicated above thus makes it possible to determine the presence of fish in the sample.

The present invention therefore also relates to a nucleotide sequence, characterized in that it is chosen from the group consisting of:
a) the reference sequences SEQ ID Nos 235 to 239, and 262 to 271,
b) the sequences complementary to each of the sequences SEQ ID Nos 235 to 239, and 262 to 271, respectively, the complementarity meaning any sequence capable of hybridizing, at a temperature of between 20 and 70° C., in saline solution at a concentration of approximately 0.5 to 1M, with any one of the sequences SEQ ID Nos 235 to 239, and 262 to 271,
c) the sequences homologous to each of the sequences SEQ ID Nos 235 to 239, and 262 to 271, and of the sequences according to b), respectively, the homology meaning any sequence, for example fragment, comprising a series of at least 5 contiguous nucleotides included in any one of said sequences and also a group of two or three nucleotides belonging to a region which has been conserved for all the species of a group under consideration, and said sequence exhibiting at least 70% identity with said any sequence.

It relates more particularly to the nucleotide sequences as defined above, and characterized in that they consist of a group of 2 to 3 nucleotides included in one of the sequences SEQ ID Nos 235 to 239 and corresponding to a region which has been conserved for all the species of a group under consideration.

It also relates to the use of the sequences defined above, that is to say characterized in that they consist of a group of 2 to 3 nucleotides included in one of the sequences SEQ ID Nos 235 to 239 and corresponding to a region which has been conserved for all the species of a group under consideration, for determining a group of original animal species in a sample liable to contain an ingredient obtained from at least one animal species belonging to said group of animal species under consideration.

These sequences, termed signature sequences, are chosen from the group consisting of the nucleotide sequence consisting of the CAA bases at positions 14689-14690-14691 of SEQ ID No. 235, the nucleotide sequence consisting of the CT bases at positions 15076-15077 of SEQ ID No. 236, the nucleotide sequence consisting of the CT bases at positions 15101-15102 of SEQ ID No. 237, the nucleotide sequence consisting of the GC bases at positions 14886-14887 of SEQ ID No. 238, and the nucleotide sequence consisting of the ATA bases at positions 14713-14726 of SEQ ID No. 239.

It relates more particularly to the nucleotide sequences as defined above, and characterized in that they consist of 1 nucleotide included in one of the sequences SEQ ID Nos 262 to 271 and corresponding to a region which has been conserved for all the species of a group under consideration.

It also relates to the use of the sequences defined above, that is to say characterized in that they consist of one nucleotide included in one of the sequences SEQ ID Nos 262 to 271 and corresponding to a region which has been conserved for all the species of a group under consideration, for determining a group of original animal species in a sample liable to contain an ingredient obtained from at least one animal species belonging to said group of animal species under consideration.

These sequences, termed signature sequences, are chosen from the group consisting of the nucleotide sequence consisting of the T base at position 14641 of SEQ ID No. 262, the nucleotide sequence consisting of the A base at position 14778 of SEQ ID No. 263, the nucleotide sequence consisting of the C base at position 15043 of SEQ ID No. 264, the nucleotide sequence consisting of the C base at position 15076 of SEQ ID No. 265, the nucleotide sequence consisting of the C base at position 15101 of SEQ ID No. 266, the nucleotide sequence consisting of the A base at position 15109 of SEQ ID No. 267, the nucleotide sequence consisting of the C base at position 15115 of SEQ ID No. 268, the nucleotide sequence consisting of the C base at position 15239 of SEQ ID No. 269, the nucleotide sequence consisting of the T base at position 14519 of SEQ ID No. 270, and the nucleotide sequence consisting of the T base at position 14717 of SEQ ID No. 271.

It also relates to a reagent for determining at least one original animal species, comprising a solid support, which may or may not be divided up, to which a nucleotide sequence chosen from the group consisting of the sequences SEQ ID Nos 235 to 239, and Nos 262 to 271, is attached.

It also relates to the method for determining a group of original animal species in a sample liable to contain an ingredient obtained from at least one species belonging to said group of animal species under consideration, characterized in that:
a) a nucleic acid fraction obtained from said sample is provided,
b) at least one reagent comprising a sequence defined above is provided,
c) the nucleic acid fraction and said reagent are brought into contact, and
d) any signal or item of information resulting from the presence of one of the signature sequences chosen from the group consisting of the nucleotide sequence consisting of the CAA bases at positions 14689-14690-14691 of SEQ ID No. 235, the nucleotide sequence consisting of the CT bases at positions 15076-15077 of SEQ ID No. 236, the nucleotide sequence consisting of the CT bases at positions 15101-15102 of SEQ ID No. 237, the nucleotide sequence consisting of the GC bases at positions 14886-14887 of SEQ ID No. 238, and the nucleotide sequence consisting of the ATA or ATG bases at positions 14713-14726 of SEQ ID No. 239, the nucleotide sequence consisting of the T base at position 14641 of SEQ ID No. 262, the nucleotide sequence consisting of the A base at position 14778 of SEQ ID No. 263, the nucleotide sequence consisting of the C base at position 15043 of SEQ ID No. 264, the nucleotide sequence consisting of the C base at position 15076 of SEQ ID No. 265, the nucleotide sequence consisting of the C base at position 15101 of SEQ ID No. 266, the nucleotide sequence consisting of the A base at position 15109 of SEQ ID No. 267, the nucleotide sequence consisting of the C base at position 15115 of SEQ ID No. 268, the nucleotide sequence consisting of the C base at position 15239 of SEQ ID No. 269, the nucleotide sequence consisting of the T base at position 14519 of SEQ ID No. 270, and the nucleotide sequence consisting of the T base at position 14717 of SEQ ID No. 271, characterizing the presence in said sample of a class of original animal species or of a group of original animal species, is determined by means of detection.

The identifying sequences can also be used as specific primers in PCR identification techniques, by mixing several primers chosen from the nucleotide sequences specific for an animal species in the presence of other species liable to be present in the media to be assayed, and in that at least one of said primers is chosen from the group consisting of the sequences SEQ ID Nos 1 to 232, and 242 to 261, and any sequences comprising at least 5 contiguous monomers included in any one of said sequences and exhibiting at least 70% identity with said any sequence.

The invention also relates to the nucleotide sequences chosen from the group consisting of the sequences SEQ ID No. 240 to SEQ ID No. 241 and SEQ ID Nos 272 to 276, and to their use as universal amplification primers, that is to say primers which can be used for detecting species in a mixture and which are sufficiently sensitive, with respect to various species, to avoid erroneous results due to the masking of certain species present in a very small proportion, because of too great a sensitivity with respect to another species liable to be present in a larger proportion. These primers are preferably used as pairs chosen from the following pairs: SEQ ID No. 240 and SEQ ID No. 241, SEQ ID No. 272 and SEQ ID No. 273, and SEQ ID No. 274 and SEQ ID No. 275.

These primers are used for carrying out the amplification steps of the methods described above, in particular when the samples comprise or are liable to contain biological material originating from species belonging to the vertebrate group.

The following examples are given by way of illustration and are in no way limiting in nature. They will make it possible to understand the invention more fully.

### EXAMPLE 1

### Detection of an Animal Species in a Sample (Table 1)

a) Preparation of the Sample

Samples originating from several animal species (mammals, birds, fish) were used in this example. The samples could be divided up into several categories:
reference samples (denoted “ref” in table 1):
reference DNA from various animal species: mammalian DNA (cattle, goat, sheep, pig, rabbit, hare, reindeer), bird DNA (ostrich, chicken, turkey, goose), fish DNA (cod, yellowfin tuna, skipjack tuna, hake, Spanish mackerel, little tunny, rainbow trout, sea trout, brook trout);
tissue samples taken in the laboratory according to a conventional protocol: oral sample from a goat, from a cat; mouse;
food samples, the exact composition and origin of which are known: blanquette of veal, beef Bourguignon, veal tongue in sauce, joint of lamb, joint of pork, chicken leg;
commercial samples (denoted “comm” in table 1) obtained from mass marketing, which are beef-based (calves' liver, beefsteak, veal chop, ground beef, joint of veal, Parmentier, Bolognaise), pork-based (ham, sausage, sausages, Chinese pork), poultry-based (ostrich steak, roast chicken, roast guinea-fowl, turkey leg, roast goose) or fish-based (European eel, salted cod fillet, canned yellowtail tuna, canned skipjack tuna, Atlantic salmon fillet, common mackerel, rainbow trout, arctic char).

All the samples are numbered (E1 to E57), and this numbering was kept in the 5 examples illustrating the invention.

Each sample is placed in a baglight® bag (Intersciences) and then blended until it is homogenized in a BagMixer®-type blender (Intersciences).

b) Lysis of 25 mg of Sample and Purification of Total DNA

The sample is lyzed and nucleic acids are purified using the Dneasy™ tissue kit (Qiagen, ref. 69504) applying the protocol recommended by Qiagen for extracting and purifying the nucleic acids from animal tissues.

c) PCR

A PCR is carried out using the Ampli Taq gold kit from Applied Biosystems according to the protocol below. The following are added to 2 μl of the total DNA suspension: the 10× gold buffer, 3.5 mM of MgCl₂, 100 μM of dNTPs (deoxyribonucleoside triphosphates), 2U of Taq gold polymerase, and 0.4 μM of the euvertebrate primers as described by Bartlett et al., in 1992 (Biotechniques Vol. 12 No. 3 pp. 408-412):
SEQ ID No. 233: 5′CCATCCAACA TCTCAGCATG ATGAAA 3′ (sequence CDL),
SEQ ID No. 234: 5′ GAAATTAATA CGACTCACTA TAGGGAGACC ACACCCCTCA GAATGATATT TGTCCTCA 3′ (sequence CBHT7, in bold: T7 polymerase promoter), in order to obtain 50 μl of final reaction volume.

A first PCR cycle of 10 minutes is carried out at 95° C., followed by 35 cycles each made up of the following 3 steps: 94° C. for 45 seconds, 50° C. for 45 seconds, 72° C. for 2 minutes. A final extension of 5 minutes at 72° C. is then carried out.

d) Verification of the Amplification

In order to verify the amplification, 5 μl of amplification product (or amplicon) are loaded onto a 1.5% agarose gel in an EDTA-Tris borate buffer. After migration for 20 minutes at 100 volts, the amplification band is visualized by staining with ethidium bromide and by illumination with ultraviolet light. The amplification is positive, as demonstrated by the presence of a band having the expected size (350 base pairs).

e) Identification of the Amplicon on a DNA Chip (Affymetrix, Santa Clara)

A biochip is synthesized on a solid support made of glass according to the method described in U.S. Pat. No. 5,744,305 (Affymetrix, Fodor et al.) using the resequencing strategy described in application WO 95/11995 (Affymax, Chee et al.) and according to the method described by A. Troesch et al. (J. Clin. Microbiol., 37(1): 49-55, 1999).

Each identifying sequence comprises 17 bases, with an interrogation position at the 10th position relative to the 3′ end of the sequence.

The analysis is carried out with the GeneChip® complete system (reference 900228, Affymetrix, Santa Clara, Calif.) which comprises the GeneArray® reader, the GeneChip® fluid station and the GeneChip® analytical software.

e.1. Transcription and Labeling of Amplicons

Due to the antisense primer CBHT7, all the amplification products have a promoter for T7 RNA polymerase. These amplicons will then serve as a matrix for a transcription reaction during which a fluorescent ribonucleotide will be incorporated.

A 2 μl aliquot is taken from the 50 μl of positive amplification product and is added to a transcription mixture containing the components of the Megascript T7 kit (Ambion, ref. 1334) and fluorescein-12-UTP (Roche, ref. 1427857). The final reaction mixture is prepared in 20 μl and the transcription reaction is carried out for 2 hours at 37° C.

e.2. Fragmentation of the Labeled Transcripts

In order to improve the hybridization conditions, the labeled transcripts are fragmented into fragments of approximately 20 nucleotides. For this, the 20 μl of labeled transcripts are subjected to the action of 30 mM imidazole (Sigma) and 30 mM manganese chloride (Merck) for 30 minutes at 65° C.

e.3. Hybridization on the DNA Chip

A 7 μl aliquot is taken from the 20 μl of labeled and fragmented transcripts and is added to 700 μl of hybridization buffer (6× SSPE (Eurobio)), 5 mM DTAB (Sigma), 3M betaine (Acros), 0.01% antifoam (ref. A80082, Sigma), and 250 μg/ml of herring sperm DNA (Gibco). This mixture is hybridized on the chip under the following conditions: 30 minutes at 40° C. After washing, the chip is scanned and the hybridization image obtained is then analyzed using the GeneChip® software (Affymetrix, Santa Clara, Calif.).

The hybridization spots make it possible to reconstitute the sequence of the amplicon, which is then compared with the reference sequences of the chip. The sequence (and therefore the species which corresponds to it) which exhibits the best percentage homology (also called “base-call”, expressed as %) with the sequence of the amplicon is selected for the identification.

e.4. Interpretation of the Results

Only part of the sequence of 350 bases is analyzed for each species. It corresponds to all or some of the identifying probes. The interpretation threshold, i.e. the level of identification, is set at a 90% base-call on the signature sequence. Below this threshold, the target, and therefore the corresponding species, is not considered to be identified.

f) Result

The DNA extracted from the food sample gives rise to an amplification product, and then to an identification on the chip. As shown in table 1, the reference samples are correctly analyzed by this technique, which also allows the detection of animal species (mammal, bird, fish) in commercial samples.

TABLE 1
Detection of an animal species in a sample

| | | | % base call | Identification |
|---|---|---|---|---|
| | | | Signature | on |
| Animal species | Nature of the sample | | sequence | chip |
| Cattle (Bos taurus) | ref | E1: bovine DNA | Bos taurus 100% | cattle |
| | | E2: bourguignon | Bos taurus 100% | cattle |
| | | E3: veal tongue | Bos taurus 100% | cattle |
| | | E4: blanquette | Bos taurus 100% | cattle |
| | | of veal | | |
| | comm | E5: veal chop | Bos taurus 95% | cattle |
| | | E6: ground beef | Bos taurus 100% | cattle |
| | | E7: joint of veal | Bos taurus 100% | cattle |
| | | E8: Parmentier | Bos taurus 100% | cattle |
| | | E9: bolognaise | Bos taurus 100% | cattle |
| | | E10: beef steak | Bos taurus 100% | cattle |
| | | E11: calves' liver | Bos taurus 100% | cattle |
| Goat (Capra hircus) | ref | E12: goat DNA | Capra hircus 100% | goat |
| | | E13: oral | Capra hircus | goat |
| | | sample | 100% | |
| Sheep (Ovis aries) | ref | E14: sheep DNA | Ovis aries | sheep |
| | | | 95.5% | |
| | | E15: joint of | Ovis aries 100% | sheep |
| | | lamb | | |
| Pig (Sus scrofa) | ref | E16: pig DNA | Sus scrofa 100% | pig |
| | | E17: joint of | Sus scrofa 100% | pig |
| | | pork | | |
| | comm | E18: ham | Sus scrofa 100% | pig |
| | | E19: sausage | Sus scrofa 100% | pig |
| | | E20: sausages | Sus scrofa 100% | pig |
| | | E21: Chinese | Sus scrofa 100% | pig |
| | | pork | | |
| Rabbit (Oryctolagus | ref | E22: rabbit DNA | Oryctolagus | rabbit |
| cuniculus) | | | cuniculus 100% | |
| Hare (Lepus | ref | E22: hare DNA | Lepus cuniculus | hare |
| cuniculus) | | | 100% | |
| Reindeer (Rangifer | ref | E23: reindeer | Rangifer | reindeer |
| tarandus) | | DNA | tarandus 100% | |
| Mouse (Mus | ref | E24: mouse | Mus musculus | mouse |
| musculus) | | | 100% | |
| Cat (Felis catus) | ref | E25: oral | Felis catus | cat |
| | | sample | 100% | |
| Ostrich (Struthio | ref | E26: ostrich | Struthio | ostrich |
| camelus) | | DNA | camelus 100% | |
| | comm | E27: ostrich | Struthio | ostrich |
| | | steak | camelus 100% | |
| Chicken (Gallus | ref | E28: chicken | Gallus gallus | chicken |
| gallus) | | DNA | 100% | |
| | | E29: Chicken | Gallus gallus | chicken |
| | | leg | 94.7% | |
| | comm | E30: roast | Gallus gallus | chicken |
| | | chicken | 100% | |
| Guinea-fowl (Numida | comm | E31: roast | Numida | guinea- |
| meleagris) | | guinea-fowl | meleagris 100% | fowl |
| Turkey (Meleagris | ref | E32: turkey DNA | Meleagris | turkey |
| gallopovo) | | | gallopovo 100% | |
| | | E33: turkey | Meleagris | turkey |
| | | joint | gallopovo 100% | |
| | comm | E34: turkey | Meleagris | turkey |
| | | legs | gallopovo 100% | |
| Goose (Anser anser) | ref | E35: goose DNA | Anser anser | goose |
| | | | 100% | |
| | comm | E36: roast | Anser anser | goose |
| | | goose | 100% | |
| European eel | comm | E37: whole fish | Anguilla | European |
| (Anguilla anguilla) | | | anguilla 100% | eel |
| Cod (Gadus morhua) | ref | E38: cod DNA | Gadus morhua | cod |
| | | | 100% | |
| | comm | E39: salted cod | Gadus morhua | cod |
| | | fillet | 100% | |
| Yellowfin tuna | ref | E40: yellowfin | Thunnus 100% | tuna |
| (Thunnus albacares) | | tuna DNA | | |
| | comm | E41: canned | Thunnus 100% | tuna |
| | | yellowfin tuna | | |
| Skipjack tuna | ref | E42: skipjack | Thunnus 94.7% | tuna |
| (Katsuwonis | | tuna DNA | | |
| pelamis) | comm | E43: canned | Thunnus 94.7% | tuna |
| | | skipjack tuna | | |
| Atlantic salmon | comm | E44: Atlantic | Salmo salar | Atlantic |
| (Salmo salar) | | salmon fillet | 100% | salmon |
| Hake (Merluccius | ref | E45: hake DNA | Merluccius | hake |
| merluccius) | | | 94.4% | |
| Spanish mackerel | ref | E46: Spanish | Scomber | Spanish |
| (Scomber japonicus) | | mackerel DNA | japonicus 100% | mackerel |
| Common mackerel | comm | E47: whole fish | Scomber | common |
| (Scomber scombrus) | | | scombrus 100% | mackerel |
| Little tunny | ref | E48: little | Euthynnus | little |
| (Euthynnus | | tunny DNA | alleteratus | tunny |
| alleteratus) | | | 100% | |
| Rainbow trout | ref | E49: rainbow | Oncorhyncus | rainbow |
| (Oncorhyncus | | trout DNA | mykiss 100% | trout |
| mykiss) | comm | E50: whole fish | Oncorhyncus | rainbow |
| | | | mykiss 100% | trout |
| Sea trout (Salmo | ref | E51: sea trout | Salmo trutta | sea trout |
| trutta fario) | | DNA | fario 100% | |
| Brook trout | ref | E52: brook | Salvenius | brook |
| (Salvenius | | trout DNA | fontinalis 100% | trout |
| fontinalis) | | | | |
| Arctic char | comm | E53: whole fish | Salvenius | Arctic |
| (Salvenius alpinus) | | | alpinus 100% | char |

### EXAMPLE 2

### Detection of Several Animal Species in a Sample (Table 2)

The experimental conditions concerning the preparation of the samples, the lysis of the samples and the purification of total DNA, the PCR, the verification of the amplification and the identification of the amplicon on a DNA chip (Affymetrix, Santa Clara) are identical to that which is described in example 1.

In this example, several animal species are simultaneously analyzed from the same sample. The analysis is carried out on:
reference samples (denoted “ref”, as in example 1) consisting of:
a mixture of DNA originating from two different animal species, in a variable proportion of each of the 2 species,
a mixture of amplicons (obtained according to the protocol of example 1), in a variable proportion of each of the two species;
commercial samples (denoted “comm”, as in example 1), derived from mass marketing, comprising several animal species in the same sample.

As presented in table 2, these results show that mixtures of species can be detected simultaneously in the same sample, whether this sample consists of a mixture of DNA, a mixture of amplicons or a commercial sample comprising several species.

TABLE 2
Detection of several animal species in a sample

| | | | |
|---|---|---|---|
| | | % base call - signature | Chip |
| Sample | Composition | sequence | identification |

1) Mixture of amplicons (after amplification)

| | | | |
|---|---|---|---|
| Beef (E1) + turkey | 80% v/v | Bos taurus 100% | cattle and |
| (E32) | 20% v/v | Meleagris gallopovo 94.1% | turkey |
| Beef (E1) + turkey | 50% v/v | Bos taurus 100% | cattle and |
| (E32) | 50% v/v | Meleagris gallopovo 100% | turkey |
| Beef (E1) + turkey | 20% v/v | Bos taurus 100% | cattle and |
| (E32) | 80% v/v | Meleagris gallopovo 100% | turkey |

2) Mixtures of DNA (before amplification)

| | | | |
|---|---|---|---|
| Pork (E16) + rabbit | 50% v/v | Oryctolagus cuniculus 100% | pig and |
| (E22) | 50% v/v | Sus scrofa 94.7% | rabbit |
| Chicken (E22) + turkey | 50% v/v | Gallus gallus 100% | chicken and |
| (E32) | 50% v/v | Meleagris gallopovo 100% | turkey |
| Beef (E1) + turkey | 99.9% v/v | Bos taurus 100% | cattle |
| (E32) | 0.1% v/v | Meleagris gallopovo 17.6% | |
| Beef (E1) + turkey | 99% v/v | Bos taurus 100% | cattle and |
| (E32) | 1% v/v | Meleagris gallopovo 95.1% | turkey |
| Beef (E1) + turkey | 90% v/v | Bos taurus 100% | cattle and |
| (E32) | 10% v/v | Meleagris gallopovo 100% | turkey |
| Beef (E1) + turkey | 50% v/v | Bos taurus 100% | cattle and |
| (E32) | 50% v/v | Meleagris gallopovo 100% | turkey |
| Beef (E1) + turkey | 1% v/v | Bos taurus 100% | cattle and |
| (E32) | 99% v/v | Meleagris gallopovo 100% | turkey |
| Beef (E1) + turkey | 0.1% v/v | Bos taurus 91% | turkey |
| (E32) | 99.9% v/v | Meleagris gallopovo 95.1% | |
| Beef (E1) + mutton | 5% v/v | Bos taurus 96.5% | cattle and |
| (E14) | 95% v/v | Ovis aries 81.1% | sheep |
| Pork (E16) + chicken | 33% v/v | Sus scrofa 96.5% | pig, chicken |
| (E22) + turkey | 33% v/v | Gallus gallus 95.6% | and turkey |
| (E32) | 33% v/v | Meleagris gallopovo 88.9% | |

3) Commercial products

| | | | |
|---|---|---|---|
| Pâté (E54) | pork + poultry | Sus scrofa 100% | pig and |
| | | Meleagris gallopovo 94.1% | turkey |
| White sausage | pork + poultry | Sus scrofa 100% | pig and |
| (E55) | | Meleagris gallopovo 94.1% | turkey |
| Kebab burger | beef + | Bos taurus 100% | cattle, goat |
| (E56) | mutton + | Capra hircus 94.1% | and sheep |
| | goat | Ovis aries 81.2% | |
| Ravioli | pork + beef | Sus scrofa 100% | cattle and |
| bolognese (E57) | | Bos taurus 95.8% | pig |
| Fromage au | cows' | Bos taurus 100% | cattle and |
| saumon [cheese | cheese + | Salmo salar 100% | salmon |
| with salmon] | salmon | | |
| (E58) | | | |
| Poultry | poultry | Gallus gallus 95% | turkey and |
| chipolata (E59) | | Meleagris gallopovo 88% | chicken |
| Torti and | pork + poultry | Sus scrofa 100% | pig and |
| fricadelles | | Gallus gallus 96.5% | chicken |
| (E60) | | | |

### EXAMPLE 3

### Detection of One or More Animal Species in Meals Intended for Animal Feed

A) Preparation of the Sample

The experimental conditions concerning the preparation of the samples are similar to those which are described in example 1. The samples are derived from meals intended for animal feed. These samples (numbered from F1 to F17) were listed beforehand in 4 categories, after analysis of the presence of bone fragments as described by Michard (Revue de l'Alimentation animale [Review of animal feed], vol. 508, pp. 43-48, 1997; reference technique).

A distinction is then made between “negative” samples, when the number of bone fragments is less than 20, “trace” samples when there are more than 20 bone fragments but a proportion of bone present in the sample of less than 0.01%, samples “to be monitored” when the proportion is between 0.01% and 1‰, and the “positive” samples when the proportion is greater than 1‰.

b) Lysis of the Sample and Purification of Total DNA

For lysing the sample and purifying the nucleic acids, the Dneasy™ tissue kit (Qiagen, ref. 69504) is used as described in example 1, along with 25 mg of meal. The technique is adapted in order to eliminate the PCR inhibitors. Specifically, these inhibitors (polyphenols, cations (Ca²⁺, Fe³⁺), traces of heavy metals, tannins, carbohydrates, salts (NaCl, nitrites)) are present in plants in considerable amounts and, as a result, in the meals intended for animal feed. This adaptation is as follows:
1- After lysis with the ATL buffer and proteinase K, chelex is added during the DNA purification step (200 μl of InstaGene™ Matrix (BIO-RAD, ref. 732-6030)).
2- After incubation for 30 minutes at 56° C., a centrifugation (5 minutes; 14 000 rpm) is carried out and the extraction is carried out as described in the Qiagen Dneasy™ tissue kit manual.
   c) PCR

A PCR is carried out using the Ampli Taq gold kit from Applied Biosystems. The following are added to 10 μl of the suspension of meal-extracted total DNA: the 10× gold buffer, 3.5 mM of MgCl₂, 100 μM of dNTPs (deoxyribonucleoside triphosphates), 2U of Taq gold polymerase, 0.4 μM of the euvertebrate primers CBL and CBHT7 as defined in example 1, in order to obtain 50 μl of final reaction volume. A first PCR cycle of 10 minutes at 95° C. is performed, followed by 35 cycles each composed of the following 3 steps: 94° C. 45 sec, 50° C. 45 sec, 72° C. 2 minutes. A final extension of 5 minutes at 72° C. is then performed.

d) Verification of the Amplification

The amplification is verified as described in example 1.

e) Identification of the Amplicon on a DNA Chip (Affymetrix, Santa Clara).

This identification step is carried out as described in example 1.

f) Result

The results obtained are given in table 3, and compared with the results obtained by means of the conventional protocol of the prior art. There is complete agreement between the 2 techniques, but with, in addition, indication of the species in the case of the invention. The invention makes it possible to detect the presence of one or more animal species in samples of meals intended for animal feed.

TABLE 3
Detection of one or more animal species in
meals intended for animal feed

| | Conventional protocol | | Protocol according |
|---|---|---|---|
| | Category | Bone fragments | to the invention |
| F1 | Negative | <20 fragments | No species detected |
| F2 | Negative | <20 fragments | No species detected |
| F3 | Negative | <20 fragments | No species detected |
| F4 | Negative | <20 fragments | No species detected |
| F5 | Trace | <0.01% | No species detected |
| F6 | Trace | <0.01% | No species detected |
| F7 | Trace | <0.01% | Pig |
| F8 | Trace | <0.01% | No species detected |
| F9 | Trace | <0.01% | Pig, mouse, cattle |
| F10 | To be monitored | 0.05% | Pig, cattle |
| F11 | To be monitored | 0.03% | Pig, cattle |
| F12 | To be monitored | 0.02% | Pig, rat, cattle |
| F13 | To be monitored | 0.01% | Pig |
| F14 | Positive | 0.23% | Pig, cattle |
| F15 | Positive | 0.23% | Cattle, pig |
| F16 | Positive | 4.70% | Cattle, pig, mouse, turkey |
| F17 | Positive | 3.50% | Cattle, mouse, pig, |
| | | | chicken |

### EXAMPLE 4

### Detection of the Class of the Species Contained in a Sample (Table 4)

The aim of this example is to obtain a technique for detecting the vertebrate class (mammals, birds, fish, etc.) of the original animal of the ingredient contained in a food sample or a sample of meal intended for animal feed.

The experimental conditions concerning a) the preparation of the sample, b) the lysis of the sample and the purification of total DNA, c) the PCR, d) the verification of the amplification and e) the identification of the amplicon on a DNA chip (Affymetrix, Santa Clara), are similar to that which is described in examples 1 and 3.

Identification of the presence of a mammal and/or fish and/or birds is determined by the presence of signatures specific for each class.

For example, for detecting the presence of mammals, use will be made of the signature sequence M1, corresponding to the sequence SEQ ID No. 235 GACACAACAA CAGC, positions 14685 to 14698 (genbank Bos taurus reference sequence; accession No. V00654). The CAA bases at positions 14689-14690-14691 (genbank Bos taurus reference sequence; accession No. V00654) are conserved for all the nucleic acid material corresponding to the predefined species making up the group that it is desired to investigate, in this case mammals. No more than 5 mutated positions are observed for the remainder of the cited signature for all the sequences making up the nucleic acid material of the group of chosen mammals. The presence of these three bases at the positions indicated above thus makes it possible to determine the presence of mammals in the sample.

Identification of the presence of birds is determined by the signatures:
O1, corresponding to the sequence SEQ ID No. 236 TCCCTAGCCT TCTC, positions 15073 to 15086 (Gallus gallus reference sequence; genbank accession No. X52392). The CT bases (positions 15076-15077) are conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of these two bases at the positions indicated above thus makes it possible to determine the presence of birds in the sample.
O2, corresponding to the sequence SEQ ID No. 237 ACACTTGCCG GAAC, positions 15098 to 15111 (Gallus gallus reference sequence; genbank accession No. X52392). The CT or CA bases (positions 15101-15102) are conserved for all the nucleic acid material corresponding to the predefined species making up the group that it is desired to investigate, in this case birds. No more than 4 mutated positions are observed for the remainder of the cited signature for all the sequences making up the nucleic acid material of the group of chosen birds. The presence of these two bases at the positions indicated above thus makes it possible to determine the presence of birds in the sample.

Identification of the presence of mammals and of birds is determined by means of the signature vi, corresponding to the sequence SEQ ID No. 238 ATAGCCACAGCATT, positions 14883 to 14896 (genbank Bos taurus reference sequence; accession No. V00654). The GC bases (at positions 14886 and 14887) are conserved for all the nucleic acid material corresponding to the predefined species making up the group that it is desired to investigate, in this case birds and mammals. No more than 4 mutated positions are observed for the remainder of the cited signature for all the sequences making up the nucleic acid material of the group of chosen birds and mammals. The presence of these two bases at the positions indicated above thus makes it possible to determine the presence of mammals and of birds in the sample.

Identification of the presence of fish is determined by the signature P1, corresponding to the sequence SEQ ID No. 239 ATAATAACCTCTTT, positions 14713 to 14726 (Gadus morhua reference sequence; genbank accession No. X99772). The ATA or ATG bases (positions 14716-14717-14718) are conserved for all the nucleic acid material corresponding to the predefined species making up the group that it is desired to investigate, in this case fish. No more than 4 mutated positions are observed for the remainder of the cited signature for all the sequences making up the nucleic acid material of the group of chosen fish. The presence of these three bases at the positions indicated above thus makes it possible to determine the presence of fish in the sample.

As shown in table 4, this technique makes it possible to detect the presence of mammals and/or birds and/or fish, whether these species are present on their own or as a mixture.

TABLE 4a
Detection of the class of species in a sample

| Samples | Signatures detected | Interpretation |
|---|---|---|
| E1: bovine DNA | V1 and M1 | mammal |
| E16: pig DNA | V1 and M1 | mammal |
| E17: joint of | V1 and M1 | mammal |
| pork | | |
| E12: goat DNA | V1 and M1 | mammal |
| E13: oral sample | V1 and M1 | mammal |
| from goat | | |
| E35: goose DNA | V1 and O1 and O2 | bird |
| E49: rainbow | P1 | fish |
| trout DNA | | |
| E51: sea trout | P1 | fish |
| DNA | | |
| Bovine/turkey | V1 and M1 and O1 and | mammal/bird |
| amplicon mixture | O2 | |
| E15: joint of | V1 and M1 | mammal |
| lamb | | |
| F9: “trace” meal | V1 and M1 | mammal |
| F1: “negative” | No positive | no |
| meal | signatures | identification |
| Meal | P1 | fish |

A variant consists in selecting not a triplet of nucleotides, but a single nucleotide representative of a given class of species.

For example, for detecting the presence of mammals, use will be made, without distinction, of:
1/ The signature sequence M2, corresponding to the sequence SEQ ID No. 262 CTAATCCTACAAATC, positions 14634 to 14648 (genbank Bos taurus reference sequence; accession No. V00654). The T base at position 14641 (genbank Bos taurus reference sequence; accession No. V00654) is conserved for all the nucleic acid material corresponding to the predefined species making up the group that it is desired to investigate, in this case mammals. No more than 5 mutated positions are observed for the remainder of the cited signature for all the sequences making up the nucleic acid material of the group of chosen mammals. The presence of this base at the position indicated above thus makes it possible to determine the presence of mammals in the sample.
2/ The signature sequence M3, corresponding to the sequence SEQ ID No. 263 AGCTTCAATGTTTTT, positions 14771 to 14785 (genbank Bos taurus reference sequence; accession No. V00654). The A base at position 14778 (genbank Bos taurus reference sequence; accession No. V00654) is conserved for all the nucleic acid material corresponding to the predefined species making up the group that it is desired to investigate, in this case mammals. No more than 5 mutated positions are observed for the remainder of the cited signature for all the sequences making up the nucleic acid material of the group of chosen mammals. The presence of this base at the position indicated above thus makes it possible to determine the presence of mammals in the sample.

For detecting birds, use may be made, without distinction, of:
1/ The signature sequence 03, corresponding to the sequence SEQ ID No. 264 CGGCCTACTACTAGC, positions 15036 to 15050 (genbank Gallus gallus reference sequence; accession No. X52392). The C base at position 15043 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
2/ The signature sequence 04, corresponding to the sequence SEQ ID No. 265 CACATCCCTAGCCTT, positions 15069 to 15083 (genbank Gallus gallus reference sequence; accession No. X52392). The C base at position 15076 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
3/ The signature sequence 05, corresponding to the sequence SEQ ID No. 266 GCCCACACTTGCCGG, positions 15094 to 15108 (genbank Gallus gallus reference sequence; accession No. X52392). The C base at position 15101 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
4/ The signature sequence 06, corresponding to the sequence SEQ ID No. 267 TTGCCGGAACGTACA, positions 15102 to 15116 (genbank Gallus gallus reference sequence; accession No. X52392). The A base at position 15109 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
5/ The signature sequence 07, corresponding to the sequence SEQ ID No. 268 GAACGTACAATACGG, positions 15108 to 15122 (genbank Gallus gallus reference sequence; accession No. X52392). The C base at position 15115 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.
6/ The signature sequence 08, corresponding to the sequence SEQ ID No. 269 TGAAACACAGGAGTA, positions 15232 to 15246 (genbank Gallus gallus reference sequence; accession No. X52392). The C base at position 15239 (genbank Gallus gallus reference sequence; accession No. X52392) is conserved for all the nucleic acid material corresponding to the predefined species making up the group that it is desired to investigate, in this case birds. No more than 5 mutated positions are observed for the remainder of the cited signature for all the sequences making up the nucleic acid material of the group of chosen birds. The presence of this base at the position indicated above thus makes it possible to determine the presence of birds in the sample.

For detecting fish, use may be made, without distinction, of:
1/ The signature sequence P2, corresponding to the sequence SEQ ID No. 270 TCAGACATCGAGACA, positions 14512 to 14526 (genbank Gadus morhua reference sequence; accession No. X99772). The T base at position 14519 (genbank Gadus morhua reference sequence; accession No. X99772) is conserved for all the nucleic acid material corresponding to the predefined species making up the group that it is desired to investigate, in this case fish. No more than 5 mutated positions are observed for the remainder of the cited signature for all the sequences making up the nucleic acid material of the group of chosen fish. The presence of this base at the position indicated above thus makes it possible to determine the presence of fish in the sample.
2/ The signature sequence P3, corresponding to the sequence SEQ ID No. 271 GTAATAATAACCTCT, positions 14710 to 14724 (genbank Gadus morhua reference sequence; accession No. X99772). The T base at position 14717 (genbank Gadus morhua reference sequence; accession No. X99772) is conserved for all the nucleic acid material corresponding to the predefined species making up the group that it is desired to investigate, in this case fish. No more than 5 mutated positions are observed for the remainder of the cited signature for all the sequences making up the nucleic acid material of the group of chosen fish. The presence of this base at the position indicated above thus makes it possible to determine the presence of fish in the sample.

As shown in table 4b, this technique makes it possible to detect the presence of mammals and/or of birds and/or of fish in a sample, in particular a food sample.

TABLE 4b
Detection of a class of species in a sample

| Samples | Signatures detected | Interpretation |
|---|---|---|
| Pork liver pâté | M3 | Mammals |
| Beef | M4 | Mammals |
| Chicken | O3 and O4 and O5 and | Birds |
| | O6 | |
| Chicken paella | O3 and O4 and O5 and | Birds |
| | O6 and O7 | |
| Spanish mackerel | P2 | Fish |
| Canned sardine | P3 | Fish |
| Fish meal | P2 | Fish |
| Fish meal | P3 | Fish |
| Fresh guinea-fowl | O1, O2, O3, O4, O5, | Birds |
| | O6, O7 and O8 | |

### EXAMPLE 5

### Universal Primers for Vertebrate Amplification (Table 5a and 5b)

The aim of the experiments presented in this example is to obtain primers which are even more sensitive than those described in the preceding examples, and more universal for detecting species in mixtures. In fact, the primers used in examples 1 to 4 are very sensitive with respect to bovine species, which can sometimes mask the presence of other species when they are present in a very small proportion.

Several pairs of primers were used in this example:
A first pair of primers comprising the following sequences
SEQ ID No. 240: 5′ GACCTCCCAG CCCCATCAAA 3′ (sequence CBL 20) and
SEQ ID No. 241: 5′ GAAATTAATA CGACTCACTA TAGGGAGACC ACACAGAATG ATATTTGTCC TCA 3′ (sequence CBHT7 20, with, in bold, the location of the T7 polymerase promoter) was chosen, initially, to increase the threshold of detection of certain species, in particular turkey or sheep, which, when they are in trace amounts in a commercial sample, can be masked by the presence of bovine species.

The technique used to obtain the identification on the chip is as described in example 1a, 1b, 1c (with the modified primers), 1d, 1e.

As shown in table 5a, the use of these new primers makes it possible to obtain, in turkey, a threshold of detection of the order of 1% compared with the primers of examples 1 to 4 where the threshold of detection was of the order of 10%. The use of these new primers also makes it possible, in commercial samples originating from mass marketing, to identify animal species, in particular sheep species, present in trace amounts, which were masked by the presence of bovine species in the preceding examples (table 5b).

TABLE 5a
Threshold of detection of turkey species in a
mixture with bovine species

| | | Detection on chip: % base call | | | |
|---|---|---|---|---|---|
| % DNA | | Primers ex. 1 to 4 | | Primers ex. 5 | |
| E1: bovine | E32: turkey | bovine | turkey | bovine | turkey |
| 100 | 0 | 100 | 5.9 | 100 | 29.4 |
| 99.9 | 0.1 | 100 | 17.6 | 100 | 41.2 |
| 99 | 1 | 100 | 76.5 | 100 | 94.1 |
| 90 | 10 | 100 | 100 | 100 | 100 |
| 50 | 50 | 100 | 100 | 100 | 100 |
| 1 | 99 | 100 | 100 | 90 | 100 |
| 0.1 | 99.9 | 100 | 100 | 60 | 100 |
| 0 | 100 | 50 | 94.1 | 26.9 | 100 |

| | | | | |
|---|---|---|---|---|
| Threshold of detection | 0.10% | 10% | 1% | 1% |

TABLE 5b
Detection of sheep species in a mixture with
other species

| | | Detection on chip: | |
|---|---|---|---|
| | | species detected | |
| Commercial | | Primers | Primers |
| products | Composition indicated | ex. 1 to 4 | ex. 5 |
| E56: Kebab | Bread, precooked ground | Bovine | Bovine |
| burger | meat (mutton, beef), | | Sheep |
| | sauce | | |
| E57: Couscous | Beef, mutton, vegetable | Bovine | Bovine |
| meatball | material | | Sheep |

Secondly, a second set of primers was chosen and used in duplex with the pair of primers described in example 1 c: when detecting animal species initially present in canned food, there may be a problem of degradation of the DNA of the animal species that it is desired to detect, in particular in the case of canned fish (for example canned tuna).

The technique used to obtain the identification on the chip is as described in examples 1a, 1b, 1d, 1e, with the exception of step 1c: 2 additional internal primers (in addition to the universal primers), which make it possible to amplify the 350 bp region in two smaller portions, are used. Several pairs of primers are studied, making it possible to amplify the 350 bp region in two regions each of between 114 and 245 bp in length, according to the primers used. Two pairs of primers were then selected for their universal nature.

A first pair of primers (used in duplex 1) comprising the following sequences:
SEQ ID No. 272: 5′ AGAIGCICCGTTTGCGTG 3′ (flanked by the T7 polymerase promoter, and I=inosine)
SEQ ID No. 273: TTCTTCTTTATCTGTITCTA (I=inosine) was chosen, initially, in order to increase the threshold of detection of certain fish species, in particular when these fish species are present in a can of food.

A second pair of primers (used in duplex 2), comprising the following sequences, was also selected:
SEQ ID No. 274: 5′ RTCICGRCARATGTG 3′ (flanked by the T7 polymerase promoter, and R=A or G, I=inosine)
SEQ ID No. 275: 5′ GTIAAYTWYGGITGACTIATCCG 3′ (M=A or C, R=A or G, Y=C or T, W=A or T, I=inosine).

In a manner comparable to that which is described in example 1c, a PCR is carried out using the Ampli Taq gold kit from Applied Biosystems (4311814). The following are added to 2 μl of the total DNA suspension: the 10× gold buffer, 3.5 mM of MgCl₂, 100 μM of dNTPs (deoxyribonucleoside triphosphates), 2U of Taq gold polymerase, 0.2 μM of the universal primers for vertebrates CBL and CBHT7 as presented in example 1c, and 0.2 μM of the primers chosen from the pairs of primers defined above (duplex 1 and duplex 2), in order to obtain 50 μl of final reaction volume. A first PCR cycle of 10 min at 95° C. is performed, followed by 35 cycles each composed of the following 3 steps: 94° C. 45 sec, 50° C. 45 sec, 72° C. 2 min. A final extension of 5 min at 72° C. is then performed.

The amplification is verified by loading 5 μl of amplification product (amplicon) onto a 1.5% agarose gel in EDTA-Tris borate. After migration for 20 min at 100V, two amplification bands are visualized by staining with ethidium bromide and by UV illumination.

The results obtained using each duplex are shown in table 5c, and compared with the results obtained by means of a “conventional” amplification using only the universal primers as described in example 1c.

TABLE 5c
Detection of several fish species in a sample
(derived from a can of food)

| | % base call - signature sequence | | |
|---|---|---|---|
| | | | Simplex |
| | | | according to |
| Sample | Duplex 1 | Duplex 2 | ex. 1 |
| Canned white tuna | 100% | 100% | 89.2% |
| (Thunnus alalunga) | | | |
| Canned Atlantic salmon | 90% | 95% | 93% |
| (Salmo salar) | | | |
| Canned flaked yellowfin | 89.5% | 94.7% | No |
| tuna (Thunnus albacares) | | | amplification |

It appears that the primers used in duplex 1 and 2 give better results and better sensitivity when it is desired to detect the presence of fish, in particular in a can of food.

It is quite evident that each primer can be used with or without the T7 promoter.

1. A method for determining an original animal species in a sample liable to contain an ingredient obtained from at least said species, characterized in that:
a) a nucleic acid fraction obtained from said sample is provided,
b) at least one reagent specific for the animal species is provided, chosen from the group consisting of
the reference sequences SEQ ID Nos 1 to 232, and Nos 242 to 261,
the sequences complementary to each of the sequences SEQ ID Nos 1 to 232, and Nos 242 to 261, respectively, the complementarity meaning any sequence capable of hybridizing, at a temperature of between 20 and 70° C., in saline solution at a concentration of approximately 0.5 to 1 M, with any one of the sequences SEQ ID Nos 1 to 232, and Nos 242 to 261,
the sequences homologous to each of the sequences SEQ ID Nos 1 to 232, and Nos 242 to 261 and of the sequences complementary to each of the sequences SEQ ID Nos 1 to 232, and Nos 242 to 261, respectively, the homology meaning any sequence, for example fragment, comprising a series of at least 5 contiguous nucleotides included in any one of said sequences, and exhibiting at least 70% identity with said any sequence,
c) the nucleic acid fraction and said reagent are brought into contact, and
d) any signal or item of information resulting from the specific reaction between said reagent and the nucleic acid fraction, characterizing the presence in said sample of said original animal species, is determined by means of detection.

2. The method as claimed in claim 1, characterized in that a set comprising a multiplicity of said reagents specific for the same original species and/or for respectively different original animal species is provided; and a multiplicity of signals or items of information characterizing the presence in said sample of the same original animal species and/or of several respectively different original animal species is determined.

3. A nucleotide sequence characterized in that it is chosen from the group consisting of:
a) the reference sequences SEQ ID Nos 1 to 232, and Nos 242 to 261,
b) the sequences complementary to each of the sequences SEQ ID Nos 1 to 232, and Nos 242 to 261, respectively, the complementarity meaning any sequence capable of hybridizing, at a temperature of between 20 and 70° C., in saline solution at a concentration of approximately 0.5 to 1 M, with any one of the sequences SEQ ID Nos 1 to 232, and Nos 242 to 261,
c) the sequences homologous to each of the sequences SEQ ID Nos 1 to 232, and Nos 242 to 261, and of the sequences according to b), respectively, the homology meaning any sequence, for example fragment, comprising a series of at least 5 contiguous nucleotides included in any one of said sequences, and exhibiting at least 70% identity with said any sequence.

4. The use of a sequence as claimed in claim 3, for determining at least one original animal species in a sample liable to contain an ingredient obtained from at least said animal species.

5. A probe for determining at least one original animal species, comprising at least one identifying nucleotide sequence as claimed in claim 3.

6. A primer for the specific amplification of a nucleic acid from an original animal species, comprising at least one identifying nucleotide sequence as claimed in claim 3.

7. A reagent for determining at least one original animal species, comprising a solid support, which may or may not be divided up, to which a nucleotide sequence as claimed in claim 3 is attached.

8. A biochip comprising a solid support comprising a developed surface, on which a multiplicity of nucleotide sequences as claimed in claim 3 is arranged and attached, according to a predetermined arrangement.

9. The method as claimed in claim 2, characterized in that the multiplicity of signals or items of information is determined with a biochip comprising a solid support comprising a developed surface, on which a multiplicity of nucleotide sequences is arranged and attached, according to a predetermined arrangement, said nucleotide sequences being chosen from the group consisting of:
a) the reference sequences SEQ ID Nos 1 to 232, and Nos 242 to 261,
b) the sequences complementary to each of the sequences SEQ ID Nos 1 to 232, and Nos 242 to 261, respectively, the complementarity meaning any sequence capable of hybridizing, at a temperature of between 20 and 70° C., in saline solution at a concentration of approximately 0.5 to 1 M, with any one of the sequences SEQ ID Nos 1 to 232, and Nos 242 to 261,
c) the sequences homologous to each of the sequences SEQ ID Nos 1 to 232, and Nos 242 to 261, and of the sequences according to b), respectively, the homology meaning any sequence, for example fragment, comprising a series of at least 5 contiguous nucleotides included in any one of said sequences, and exhibiting at least 70% identity with said any sequence.

10. A nucleotide sequence characterized in that it is chosen from the group consisting of:
a) the reference sequences SEQ ID Nos 235 to 239, and 262 to 271,
b) the sequences complementary to each of the sequences SEQ ID Nos 235 to 239, and 262 to 271, respectively, the complementarity meaning any sequence capable of hybridizing, at a temperature of between 20 and 70° C., in saline solution at a concentration of approximately 0.5 to 1M, with any one of the sequences SEQ ID Nos 235 to 239, and 262 to 271,
c) the sequences homologous to each of the sequences SEQ ID Nos 235 to 239, and 262 to 271, and of the sequences according to b), respectively, the homology meaning any sequence, for example fragment, comprising a series of at least 5 contiguous nucleotides included in any one of said sequences and also a group of two or three nucleotides belonging to a region which has been conserved for all the species of a group under consideration, and said sequence exhibiting at least 70% identity with said any sequence.

11. A nucleotide sequence characterized in that it consists of a group of 1 to 3 nucleotides included in one of the sequences as claimed in claim 10 and corresponding to a region which has been conserved for all the species of a group under consideration.

12. The nucleotide sequence as claimed in claim 11, characterized in that it consists of the CAA bases at positions 14689-14690-14691 of SEQ ID No. 235 or the CT bases at positions 15076-15077 of SEQ ID No. 236 or the CT bases at positions 15101-15102 of SEQ ID No. 237 or the GC bases at positions 14886-14887 of SEQ ID No. 238 or the ATA bases at positions 14713-14726 of SEQ ID No. 239.

13. The nucleotide sequence as claimed in claim 11, characterized in that it consists of the T base at position 14641 of SEQ ID No. 262 or the A base at position 14778 of SEQ ID No. 263 or the C base at position 15043 of SEQ ID No. 264, or the C base at position 15076 of SEQ ID No. 265, or the C base at position 15101 of SEQ ID No. 266, or the A base at position 15109 of SEQ ID No. 267, or the C base at position 15115 of SEQ ID No. 268, or the C base at position 15239 of SEQ ID No. 269, or of the nucleotide sequence consisting of the T base at position 14519 of SEQ ID No. 270 or the T base at position 14717 of SEQ ID No. 271.

14. A reagent for determining at least one original animal species, comprising a solid support, which may or may not be divided up, to which a nucleotide sequence as claimed in claim 10 is attached.

15. A method for determining a group of original animal species in a sample liable to contain an ingredient obtained from at least one species belonging to said group of animal species under consideration, characterized in that:
a) a nucleic acid fraction obtained from said sample is provided,
b) at least one reagent comprising a sequence as claimed in claim 10 is provided,
c) the nucleic acid fraction and said reagent are brought into contact, and
d) any signal or item of information resulting from the presence of a sequence consisting of a group of 1 to 3 nucleotides, characterizing the presence in said sample of a group of original animal species, is determined by means of detection, wherein the sequence consisting of a group of 1 to 3 nucleotides corresponds to a region that has been conserved for all the species of a group under consideration and is included in one of the sequences chosen from the group consisting of:
a) the reference sequences SEQ ID Nos 235 to 239, and 262 to 271,
b) the sequences complementary to each of the sequences SEQ ID Nos 235 to 239, and 262 to 271, respectively, the complementarity meaning any sequence capable of hybridizing, at a temperature of between 20 and 70° C., in saline solution at a concentration of approximately 0.5 to 1M, with any one of the sequences SEQ ID Nos 235 to 239, and 262 to
c) the sequences homologous to each of the sequences SEQ ID Nos 235 to 239, and 262 to 271, and of the sequences according to b), respectively, the homology meaning any sequence, for example fragment, comprising a series of at least 5 contiguous nucleotides included in any one of said sequences and also a group of two or three nucleotides belonging to a region which has been conserved for all the species of a group under consideration, and said sequence exhibiting at least 70% identity with said any sequence.

16. The use of the sequences defined in claim 11, for determining a group of original animal species in a sample liable to contain an ingredient obtained from at least one animal species belonging to said group of animal species under consideration.

17. A method for determining a group of original animal species in a sample liable to contain an ingredient obtained from at least one species belonging to said group of animal species under consideration, characterized in that:
a) a nucleic acid fraction obtained from said sample is provided,
b) the nucleotide sequence(s) characteristic of the group of animal species to be determined is (are) identified,
c) at least one reagent comprising a sequence identified in step b) is provided,
c) the nucleic acid fraction and said reagent are brought into contact, and
d) any signal or item of information resulting from the presence of one of the sequences as claimed in claim 11, characterizing the presence in said sample of a group of original animal species, is determined by means of detection.
